(19) United States
(12) Patent Application Publication
(10) Pub. No.: US 2024/0013854 A1
(43) Pub. Date: Jan. 11, 2024

(54) SYSTEMS AND METHODS FOR ENGINEERING PROTEIN ACTIVITY

(71) Applicant: Aether Biomachines, Inc., Menlo Park, CA (US)

(72) Inventors: Stylianos Kyriacou, Redwood City, CA (US); Pavle Jeremic, Stanford, CA (US); Charmaine Chia, Menlo Park, CA (US); Inhee Park, San Jose, CA (US); Louis A. Clark, San Francisco, CA (US); Christian Fitzgerald Clough, London (GB)

(21) Appl. No.: 18/369,788

(22) Filed: Sep. 18, 2023

Related U.S. Application Data
(63) Continuation of application No. PCT/US2023/060334, filed on Jan. 9, 2023.
(60) Provisional application No. 63/307,957, filed on Feb. 8, 2022.
(60) Provisional application No. 63/298,012, filed on Jan. 10, 2022.

(51) Int. Cl. G16B 15/20; G16B 40/20; G06F 30/27

(52) U.S. Cl. CPC G16B 15/20; G16B 40/20; G06F 30/27

(57) ABSTRACT

The present disclosure provides systems and methods for engineering protein activity. In an aspect, described herein are new predictive models that predict protein activity from its amino acid sequence (e.g., using a trained machine learning model and a physics-based simulation model). In another aspect, new prescriptive models identify one or more candidate proteins (e.g., for use in the predictive model) that can use a multi-objective search and optimization algorithm. In another aspect, predictive and prescriptive models can be combined, e.g., with the addition of high-throughput laboratory data which has been by analyzed by a descriptive model. In another aspect, described herein are systems and methods for high-throughput automated expression of a plurality of proteins. In another aspect, described herein are systems and methods for high-throughput automated functional screening of a plurality of proteins. In another aspect, described herein are systems and methods for quantifying an amount of cellular proliferation.

### CROSS-REFERENCE TO RELATED APPLICATIONS

This application is a continuation application of, and claims priority to, PCT application PCT/US2023/060334 filed Jan. 9, 2023, which claims the benefit of U.S. Provisional Application 63/298,012 filed on Jan. 10, 2022 and U.S. Provisional Application 63/307,957 filed on Feb. 8, 2022. The disclosure of the foregoing applications are incorporated here by reference.

### BACKGROUND

Proteins can be useful for catalyzing chemical reactions and as precise binding reagents (e.g., for diagnostics, therapeutics, detection, or separations). Enzymatic catalysis can replace traditional chemical synthesis which often relies on the use of petrochemical-based solvents, hazardous reagents, and metal-based chemical catalysts to bring about chemical transformations. In contrast, enzymes are nonhazardous and nontoxic, biodegradable, operate in aqueous and non-aqueous solvents, and are completely renewable, as they are produced safely and inexpensively through biological processes. In addition, they can function with exquisite selectivity and can in principle allow the synthesis of products inaccessible by traditional chemical synthesis, as well as the use of alternative raw materials that could dramatically lower infrastructure and operating costs. When used as binding reagents (e.g., as an antibody), proteins can offer similar advantage with regard to selectivity in binding the desired ligand or epitope and not closely related ligands or epitopes. This can result in improved precision of detection, treatments with reduced side effects, or selective enrichment of a product.

While previous advances in engineering of protein activity have brought improvements in many proteins compared to wild-type (i.e., a variant of the protein found in nature), previous improvements typically take several months to complete, achieve only modest improvements, and have unpredictable results. Furthermore, previous approaches typically require an initial protein having at least some activity on the desired substrate, and are therefore limited to improving a protein rather than engineering novel activity. There is a need for fundamentally new systems and methods for high-throughput protein design.

Furthermore, there is a need for improved systems and methods for high-throughput protein expression and screening. Such systems and methods would shorten the development time for such proteins, as well as screen more protein variants in order to discover proteins with novel activities and/or improved binding.

### SUMMARY

The systems and methods describe herein satisfy the need for new and improved systems and methods for engineering protein activity. The systems and methods redefine protein engineering as a search and optimization problem, which is solved by the use of the computational and laboratory methods described herein which use machine learning and artificial intelligence. These systems and methods can balance the multiple or contradictory objectives (e.g., enzymatic activity vs selectivity), the presence of certain constraints (e.g., protein stability at industrial or biological conditions), and the complex mapping between sequence and the metrics of interest. The systems and methods are also able to effectively leverage data from an earlier use of the methods to accelerate future uses of the method (i.e., through transfer learning).

New predictive models have been developed here to predict protein activity from its amino acid sequence (e.g., using a trained machine learning model and a physics-based simulation model). New prescriptive models have been developed to identify one or more candidate proteins (e.g., for use in the predictive model) that can use a multi-objective search and optimization algorithm. Predictive and prescriptive models can also be combined, e.g., with the addition of high-throughput laboratory data which has been by analyzed by a descriptive model.

The systems and methods described herein can be trained on laboratory data and have their results and suggestions validated by laboratory data. However, prior methods of enzyme synthesis and detection of reaction products are too costly and laborious to match the scale of the computational methods described herein (e.g., provide too little, too costly data). Therefore, the present disclosure satisfies the need for protein expression and screening at high-scale and with high-throughput. In some embodiments, the system can express at least 2,000 proteins in a day or screen 10,000 reaction products in a day. This can be achieved by automating and integrating modules that perform various functions in novel ways as described herein. In synergistic combination, these modules allow miniaturized enzyme expression and testing with reagent volumes an order of magnitude smaller than prior methods. This also considerably reduces consumable costs per experiment. For example, liquid dispensing technologies that operate from nanoliter to milliliter volumes and do not use pipette tips help to provide scale and cost savings. Software translates lab work into data via integration with a laboratory information management system (LIMS), a central repository for storing and accessing data. Together, these modules allow rapid design, sequence and testing of proteins to catalyze many different chemical reactions and/or bind many different ligands.

In an aspect, provided herein is a method for enzyme design. The method can include proposing a plurality of candidate proteins using a prescriptive model that comprises a multi-objective search and optimization algorithm; for each of the plurality of candidate proteins, predicting an enzymatic activity on a substrate using a predictive model that combines a machine learning algorithm and a physics-based simulation; selecting and expressing at least some of the candidate proteins; and measuring an activity on the substrate for the expressed candidate proteins.

In some embodiments, the method further comprises using the measured activity to propose another plurality of candidate proteins using the prescriptive model.

In some embodiments, the method further comprises using the measured activity to train or improve the prescriptive model.

In some embodiments, the method further comprises using the measured activity to train or improve the machine learning algorithm of the predictive model.

In some embodiments, the candidate proteins are selected for expression based at least in part on the predicted activity on the substrate.

In some embodiments, the selected candidate proteins are expressed in E. coli.

In some embodiments, a quantity of E. coli that is cultured is measured using image analysis of pelleted E. coli cells.

In some embodiments, the E. coli are lysed to release the expressed proteins.

In some embodiments, the expressed proteins are enriched.

In some embodiments, the expressed proteins are contacted with the substrate.

In some embodiments, a concentration of the substrate and/or a reaction product are measured to determine an activity.

In some embodiments, an activity for each candidate protein is predicted and measured for a plurality of substrates.

In some embodiments, at least one of the plurality of substrates is a desired substrate.

In some embodiments, at least two of the plurality of substrates have substantially similar structures.

In some embodiments, at least two of the plurality of substrates have substantially dis-similar structures.

In another aspect, provided herein is a method for evaluating a candidate protein. The method can include, by one or more computing devices: obtaining an amino acid sequence associated with the candidate protein; inputting the amino acid sequence into a predictive model to obtain a predicted activity on a substrate, wherein the predictive model comprises a trained machine learning model and a physics-based simulation model; and evaluating the candidate protein based on the predicted activity.

In some embodiments, the activity is an enzymatic activity or a binding activity on a substrate.

In some embodiments, the amino acid sequences associated a plurality of candidate proteins are obtained using a prescriptive model and input into the predictive model.

In some embodiments, the prescriptive model comprises a multi-objective search and optimization algorithm.

In some embodiments, said evaluating comprises selecting one or more candidate proteins based at least partially on the predicted activity.

In some embodiments, the one or more candidate proteins are selected for measurement of activity.

In some embodiments, the predictive model is further configured to receive a measured activity for the candidate protein.

In some embodiments, the activity is measured by expressing the candidate protein and exposing the expressed protein to the substrate.

In some embodiments, the predictive model is configured to be trained using the measured activity.

In some embodiments, machine learning model is configured to be trained using a combination of stochastic and deterministic optimization methods.

In some embodiments, the training increases accuracy of the predictive model for predicting activity on the substrate.

In some embodiments, the increased accuracy of prediction is achieved for a candidate protein having at least 5 amino acid differences from any protein having a known activity on the substrate.

In some embodiments, the predictive model is configured to be tuned using a measured activity.

In some embodiments, the tuning is performed by hot-starting from a version of the predictive model that has not been tuned using the measured activity.

In some embodiments, the tuning takes experimental error into account.

In some embodiments, the predicted activity comprises enzymatic activity and selectivity of converting the substrate into a desired product.

In some embodiments, the predictive model is further configured to predict a stability or toxicity of the candidate protein.

In some embodiments, the physics-based model is configured to provide features for the machine learning model of the predictive model.

In some embodiments, the features are a set of physicochemical attributes that are assigned per amino acid residue, along with their corresponding structure-based vicinity graph.

In some embodiments, the features are a set of physicochemical attributes that are assigned per atom, along with their corresponding structure-based vicinity graph.

In some embodiments, the structure-based vicinity graph is a coded version of a folded structure of the candidate protein.

In some embodiments, the features comprise a folded structure for the candidate protein, a location at which the substrate is expected to dock with the candidate protein, expected vibrations of the substrate around a docked location of the candidate protein, an expected enzymatic reaction between the substrate and the candidate protein, or any combination thereof.

In some embodiments, the physics-based model is configured to compute enzyme and substrate relative positions.

In some embodiments, the physics-based model is configured to provide training patterns for the machine learning model of the predictive model.

In some embodiments, the physics-based model constrains the machine learning model of the predictive model to a physical solution space.

In some embodiments, the physics-based model comprises a molecular docking algorithm, a molecular dynamics algorithm, a quantum molecular dynamics algorithm, or any combination thereof.

In some embodiments, the molecular docking algorithm is configured to find a likely location for the substrate to dock with the candidate protein.

In some embodiments, the molecular dynamics algorithm is configured to predict vibrations of the substrate around a docked location of the candidate protein.

In some embodiments, the quantum molecular dynamics algorithm is configured to simulate an enzymatic reaction between the substrate and the candidate protein.

In some embodiments, the machine learning model includes a convolutional neural network (CNN), a random forest (RF), a multilayer perceptron (MLP), a radial basis function network (RBFN), a graph neural network (GNN), or any combination thereof.

In some embodiments, machine learning model is configured to predict a folded structure for the candidate protein based on the amino acid sequence of the candidate protein.

In some embodiments, the folded structure is used in the physics-based model.

In another aspect, provided herein is a method for identifying one or more candidate proteins. The method can include, by one or more computing devices: obtaining a plurality of candidate proteins using a prescriptive model comprising a multi-objective search and optimization algorithm; inputting each candidate protein of the plurality of candidate proteins into a predictive model to obtain a predicted activity on a substrate; and selecting one or more candidate proteins from the plurality of candidate proteins at least partially based on the predicted activities.

In some embodiments, an amino acid sequence of the candidate protein is obtained from the prescriptive model.

In some embodiments, an amino acid sequence of the candidate protein is input into the predictive model.

In some embodiments, the activity is an enzymatic activity or a binding activity on a substrate.

In some embodiments, the predictive model comprises a trained machine learning model and a physics-based simulation model.

In some embodiments, the one or more candidate proteins are selected for measurement of activity.

In some embodiments, the prescriptive model is further configured to receive measured activity for the candidate protein.

In some embodiments, the activity is measured by expressing the subset of candidate proteins and exposing each of the expressed proteins to the substrate.

In some embodiments, the prescriptive model is configured to be trained using the measured activity.

In some embodiments, the plurality of candidate proteins obtained using the prescriptive model is a sub-set of all of the proteins considered by and/or available for consideration by the prescriptive model.

In some embodiments, a candidate protein is obtained from the prescriptive model based at least in part on its expected activity on the substrate.

In some embodiments, a candidate protein obtained from the prescriptive model has at least 3 amino acid differences from any protein having a known activity on the substrate.

In some embodiments, an activity on the substrate has not previously been measured for the candidate proteins obtained from the prescriptive model.

In some embodiments, a candidate protein obtained from the prescriptive model is expected to have an optimal or near-optimal enzymatic quality in relation to the multiple objectives.

In some embodiments, the multiple objectives comprise enzymatic activity, selectivity, stability, toxicity, size, novelty, or any combination thereof.

In some embodiments, the enzymatic quality is on or in proximity to a pareto frontier.

In some embodiments, the prescriptive model is based at least partially on a combination of stochastic and deterministic optimization methods.

In some embodiments, the stochastic method is configured to randomly vary an amino acid identity at one or more positions of the candidate protein sequence.

In some embodiments, the deterministic method is configured to compute and select an amino acid identity for one or more positions of the candidate protein sequence.

In some embodiments, the prescriptive model comprises a meta-model-assisted evolutionary algorithm.

In some embodiments, the meta-modeling algorithm is configured to predict an enzymatic activity for a candidate protein based at least in part on the amino acid sequence of the candidate protein.

In some embodiments, the meta-modeling algorithm does not use a structure or predicted structure of the candidate protein.

In some embodiments, the meta-modeling algorithm comprises a machine learning algorithm.

In some embodiments, the machine learning algorithm includes a random forest (RF), a multilayer perceptron (MLP), a radial basis function network (RBFN), a graph neural network (GNN), or any combination thereof.

In some embodiments, the prescriptive model is further configured to use a hierarchical optimization algorithm.

In some embodiments, the prescriptive model is configured to use levels of at least two of a docking model, a quantum-mechanical model, a machine learning model, and a molecular dynamics model.

In some embodiments, the plurality of candidate proteins is obtained by running the prescriptive model using a distributed computation method.

In some embodiments, the predicted activity comprises enzymatic activity and selectivity of converting the substrate into a desired product.

In some embodiments, the predictive model is further configured to predict a stability or toxicity of the folded protein at a given set of conditions.

In another aspect, provided herein is a method for obtaining one or more candidate proteins. The method can comprise, by one or more computing devices: receiving an initial set of candidate proteins; and obtaining the one or more candidate proteins by performing, based on the initial set of candidate proteins, one or more iterations of an evolutionary algorithm which utilizes problem-specific evolution operators, each iteration comprising: evaluating a current set of candidate proteins; and based on the evaluation, updating the current set of candidate proteins.

In some embodiments, a subsequent set of initial candidate proteins are generated and received by the evolutionary algorithm, based at least in part on the current set of candidate proteins.

In some embodiments, the problem-specific evolution operators are based at least in part on physics.

In some embodiments, the problem-specific evolution operators are based at least in part on amino acid sequence.

In some embodiments, the problem-specific evolution operators are based at least in part on protein structure.

In some embodiments, the problem-specific evolution operators comprise multiple sequence analysis (MSA).

In some embodiments, the evolutionary algorithm comprises a multi-objective search and optimization algorithm.

In some embodiments, the amino acid sequences of the initial set of candidate proteins are received by the evolutionary algorithm.

In some embodiments, the amino acid sequences of the candidate proteins are obtained from the evolutionary algorithm.

In some embodiments, the method further comprises inputting the current set of candidate proteins into a predictive model, which predictive model predicts activity for each of the candidate proteins on a substrate.

In some embodiments, the predictive model comprises a trained machine learning model and a physics-based simulation model.

In some embodiments, the activity is an enzymatic activity or a binding activity on a substrate.

In some embodiments, the predictive model selects one or more candidate proteins for measurement of activity.

In some embodiments, the evolutionary algorithm is assisted by a meta-modeling algorithm.

In some embodiments, the meta-modeling algorithm comprises a machine learning algorithm.

In some embodiments, the meta-modeling algorithm is configured to predict an activity for a candidate protein.

In some embodiments, the predicted activity comprises enzymatic activity and selectivity of converting the substrate into a desired product.

In some embodiments, the predictive model is further configured to predict a stability or toxicity of the folded protein at a given set of conditions.

In some embodiments, the prediction of activity is based at least in part on the amino acid sequence of the candidate protein.

In some embodiments, the meta-modeling algorithm does not use a structure or predicted structure of the candidate protein.

In some embodiments, the machine learning algorithm includes a random forest (RF), a multilayer perceptron (MLP), a radial basis function network (RBFN), a graph neural network (GNN), or any combination thereof.

In some embodiments, the meta-modeling algorithm is further configured to receive measured activity for the candidate protein.

In some embodiments, the meta-modeling algorithm is configured to be trained using the measured activity.

In some embodiments, the activity is measured by expressing the current set of candidate proteins and exposing each of the expressed proteins to the substrate.

In some embodiments, at least one of the current sets of candidate proteins have at least 3 amino acid differences from any protein having a known activity on the substrate.

In some embodiments, an activity on the substrate has not previously been measured for the current set of candidate proteins.

In some embodiments, the current set of candidate proteins is expected to have an optimal or near-optimal enzymatic quality in relation to the multiple objectives.

In some embodiments, the multiple objectives comprise enzymatic activity, selectivity, stability, toxicity, size, novelty, or any combination thereof.

In some embodiments, the enzymatic quality is on or in proximity to a pareto frontier.

In some embodiments, the evolutionary algorithm is based at least partially on a combination of stochastic and deterministic optimization methods.

In some embodiments, the stochastic method is configured to randomly vary an amino acid identity at one or more positions of the candidate protein sequence.

In some embodiments, the deterministic method is configured to compute and select an amino acid identity for one or more positions of the candidate protein sequence.

In some embodiments, the evolutionary algorithm is further configured to use a hierarchical optimization algorithm.

In some embodiments, the evolutionary algorithm is configured to use levels of at least two of a docking model, a quantum-mechanical model, a machine learning model, and a molecular dynamics model.

In some embodiments, the current set of candidate proteins is obtained by running the evolutionary algorithm using a distributed computation method.

In another aspect, provided herein is a system for enzyme design. The system can comprise: one or more processors; one or more memories; and one or more programs, wherein the one or more programs are stored in the one or more memories and configured to be executed by the one or more processors, the one or more programs including instructions for: proposing a plurality of candidate proteins using a prescriptive model that comprises a multi-objective search and optimization algorithm; for each of the plurality of candidate proteins, predicting an enzymatic activity on a substrate using a predictive model that combines a machine learning algorithm and a physics-based simulation; selecting and expressing at least some of the candidate proteins; and measuring an activity on the substrate for the expressed candidate proteins.

In another aspect, provided herein is a system for evaluating a candidate protein, comprising: one or more processors; one or more memories; and one or more programs, wherein the one or more programs are stored in the one or more memories and configured to be executed by the one or more processors, the one or more programs including instructions for: obtaining an amino acid sequence associated with the candidate protein; inputting the amino acid sequence into a predictive model to obtain a predicted activity on a substrate, wherein the predictive model comprises a trained machine learning model and a physics-based simulation model; and evaluating the candidate protein based on the predicted activity.

In another aspect, provided herein is a system for identifying one or more candidate proteins, comprising: one or more processors; one or more memories; and one or more programs, wherein the one or more programs are stored in the one or more memories and configured to be executed by the one or more processors, the one or more programs including instructions for: obtaining a plurality of candidate proteins using a prescriptive model comprising a multi-objective search and optimization algorithm; inputting each candidate protein of the plurality of candidate proteins into a predictive model to obtain a predicted activity on a substrate; and selecting one or more candidate proteins from the plurality of candidate proteins at least partially based on the predicted activities.

In another aspect, provided herein is a system for obtaining one or more candidate proteins, comprising: one or more processors; one or more memories; and one or more programs, wherein the one or more programs are stored in the one or more memories and configured to be executed by the one or more processors, the one or more programs including instructions for: receiving an initial set of candidate proteins; and obtaining the one or more candidate proteins by performing, based on the initial set of candidate proteins, one or more iterations of an evolutionary algorithm which utilizes problem-specific evolution operators, each iteration comprising: evaluating a current set of candidate proteins; and based on the evaluation, updating the current set of candidate proteins.

In another aspect, provided herein is a non-transitory computer-readable storage medium storing one or more programs for enzyme design, the one or more programs comprising instructions, which when executed by one or more processors of an electronic device, cause the electronic device to: propose a plurality of candidate proteins using a prescriptive model that comprises a multi-objective search and optimization algorithm; for each of the plurality of candidate proteins, predict an enzymatic activity on a substrate using a predictive model that combines a machine learning algorithm and a physics-based simulation; select and expressing at least some of the candidate proteins; and measure an activity on the substrate for the expressed candidate proteins.

In another aspect, provided herein is a non-transitory computer-readable storage medium storing one or more programs for evaluating a candidate protein, the one or more programs comprising instructions, which when executed by one or more processors of an electronic device, cause the electronic device to: obtain an amino acid sequence associated with the candidate protein; input the amino acid sequence into a predictive model to obtain a predicted activity on a substrate, wherein the predictive model comprises a trained machine learning model and a physics-based simulation model; and evaluate the candidate protein based on the predicted activity.

In another aspect, provided herein is a non-transitory computer-readable storage medium storing one or more programs for identifying one or more candidate proteins, the one or more programs comprising instructions, which when executed by one or more processors of an electronic device, cause the electronic device to: obtain a plurality of candidate proteins using a prescriptive model comprising a multi-objective search and optimization algorithm; input each candidate protein of the plurality of candidate proteins into a predictive model to obtain a predicted activity on a substrate; and select one or more candidate proteins from the plurality of candidate proteins at least partially based on the predicted activities.

In another aspect, provided herein is a non-transitory computer-readable storage medium storing one or more programs for obtaining one or more candidate proteins, the one or more programs comprising instructions, which when executed by one or more processors of an electronic device, cause the electronic device to: receive an initial set of candidate proteins; and obtain the one or more candidate proteins by performing, based on the initial set of candidate proteins, one or more iterations of an evolutionary algorithm which utilizes problem-specific evolution operators, each iteration comprising: evaluating a current set of candidate proteins; and based on the evaluation, updating the current set of candidate proteins.

In another aspect, provided herein is a method for high-throughput automated expression of a plurality of proteins, the method comprising: providing a colony plate having colonies of cells dispersed thereon, wherein the cells are capable of expressing one of a plurality of proteins; robotically inoculating a plurality of cultures from the colonies of cells, wherein the cultures are arrayed in wells of a culture plate; incubating the culture plate at conditions suitable for growing the cells in the cultures; and obotically harvesting the cells from the cultures to provide cell pellets, wherein a single person is capable of incubating at least 2,000 cultures in a day.

In another aspect, provided herein is a method for high-throughput automated functional screening of proteins, the method comprising: providing a plurality of proteins; robotically combining each of the plurality of proteins with a substrate on a reaction array,

incubating the reaction array to produce a plurality of reaction products, which incubation is at conditions that are suitable for the proteins to transform the substrate into a reaction product; robotically transferring the reaction products to a detection array; and detecting the reaction products on the detection array, wherein a single person is capable of detecting at least 10,000 reaction products in a day.

In another aspect, provided herein is a method for quantifying an amount of cellular proliferation, the method comprising: centrifuging a plate containing an array of cell cultures to provide an array of cell pellets in a solution; optionally removing the solution from the plate; imaging the plate to provide an image; from the image, detecting data associated with the cell pellets, which data comprises a location of each cell pellet; and quantifying a mass or number of cells in each cell pellet using the data associated with the cell pellets, wherein the method is capable of quantifying a mass or number of cells in at least 96 cell pellets from a single image.

In another aspect, provided herein is a method for high-throughput automated expression and functional screening of proteins, the method comprising: providing a colony plate having colonies of cells dispersed thereon, wherein the cells are capable of expressing one of a plurality of proteins; robotically inoculating a plurality of cultures from the colonies of cells, wherein the cultures are arrayed in wells of a culture plate; incubating the culture plate at conditions suitable for growing the cells in the cultures; robotically harvesting the cells from the cultures to provide cell pellets; imaging the cell pellets and using the image and a trained machine learning algorithm to quantify a mass or quantity of cells in the cell pellets; robotically combining each of the plurality of proteins from the cell pellets with a substrate in a reaction array; incubating the reaction array to produce a plurality of reaction products, which incubation is at conditions that are suitable for the proteins to transform the substrate into a reaction product; robotically transferring the reaction products to a detection array; and detecting the reaction products on the detection array, wherein a single person is capable of detecting at least 10,000 reaction products in a day.

In another aspect, provided herein is a system for high-throughput automated expression of a plurality of proteins, the system comprising: a reagent dispenser configured to dispense media into wells of a culture plate; a colony picker configured to pick colonies of cells from a colony plate and inoculate a plurality of cultures in wells of the culture plate; an incubator configured to incubate the culture plate at conditions suitable for growing the cells; a harvester configured to enrich the cells from the culture plate to provide cell pellets; and a plate transfer module configured to move the culture plate between the reagent dispenser, the colony picker, the incubator, and the centrifuge, wherein the system is capable of incubating at least 2,000 cultures in a day.

In another aspect, provided herein is a system for high-throughput automated functional screening of proteins, the system comprising: an incubator configured to incubate a plurality of proteins with a substrate on a reaction array to produce a plurality of reaction products; a detection module configured to detect the reaction products on a detection array; one or more liquid handlers configured to lyse cells containing the protein, aliquot the protein onto the reaction array, aliquot the substrate onto the reaction array, and/or transfer some of the reaction products from the reaction array to the detection array; and a plate transfer module configured to move the reaction array and/or the detection array between the incubator, the detection module, and the one or more liquid handlers, wherein the system is capable of detecting at least 10,000 reaction products in a day.

In another aspect, provided herein is a system for high-throughput automated functional screening of proteins, the system comprising: a colony picker configured to pick colonies of cells from a colony plate and inoculate a plurality of cultures in wells of a culture plate; one or more incubators configured to incubate the culture plate at conditions suitable for growing the cells and expressing a protein in the cells, and incubate a plurality of proteins with a substrate on a reaction array to produce a plurality of reaction products; a harvestor configured to harvest the cells from the culture plate to provide cell pellets; a detection module configured to detect the reaction products on a detection array; one or more liquid handlers configured to dispense media into wells of a culture plate, lyse cells containing the protein, aliquot the protein onto the reaction array, aliquot the substrate onto the reaction array, and/or transfer some of the reaction products from the reaction array to the detection array; and a plate transfer module configured to move the culture plate, the reaction array and/or the detection array between the one or more liquid handlers, the colony picker, the one or more incubators, the centrifuge and the detection module.

### DESCRIPTION OF THE FIGURES

FIG. 1 depicts an example of the methods described herein for engineering protein activity.

FIG. 2 depicts an example of the relationship between laboratory measurement and the descriptive, predictive, and prescriptive models described herein for engineering protein activity

FIG. 3 shows an example of the systems and methods described herein using a trained machine learning model without a physics-based simulation model.

FIG. 4 depicts an example of a predictive model converting an input into a predicted activity, as described herein.

FIG. 5 depicts an example of a physics-based simulation being used according to the present systems and methods to predict a protein activity.

FIG. 6 depicts an example of using more than one physics-based simulation being used in combination to predict a binding energy.

FIG. 7 depicts examples of predictive modules as described herein.

FIG. 8 depicts examples of predictive modules as described herein.

FIG. 9 depicts examples of predictive modules as described herein.

FIG. 10 depicts examples of predictive modules as described herein.

FIG. 11 depicts an example of a prescriptive model that includes a metamodel.

FIG. 12 depicts an example of a hierarchical model that can be used in the prescriptive model.

FIG. 13 depicts an example of a prescriptive model described herein having an metamodel assisted evolutionary algorithm and a hierarchical metamodel assisted evolutionary algorithm.

FIG. 14 shows an example of a prescriptive model that uses neighbors with respect to product similarity, substrate similarity, and/or active site similarity as parents.

FIG. 15 shows an example of a prescriptive model that uses multiple approaches.

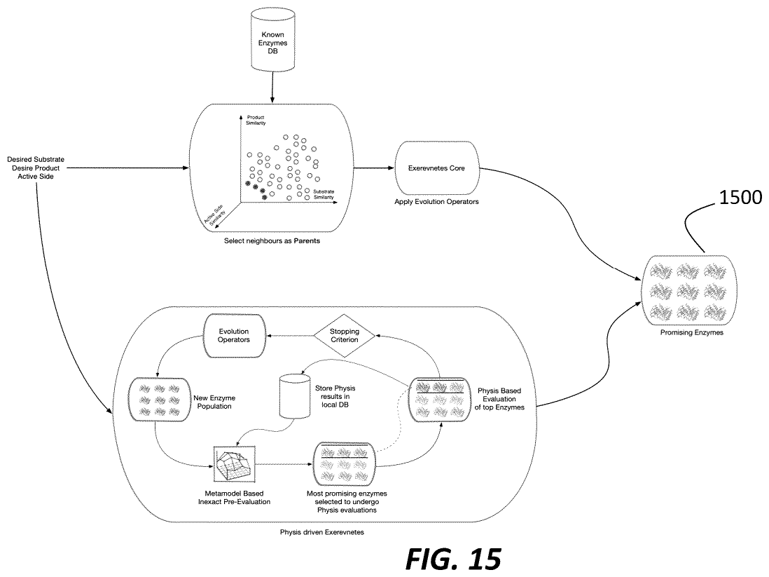

FIG. 16 depicts an example of the method described herein for expression of proteins.

FIG. 17 depicts an example of the system described herein for expression of proteins.

FIG. 18 depicts an example of the method described herein for quantification of cellular proliferation.

FIG. 19 depicts an example of the method described herein for screening of proteins.

FIG. 20 depicts an example of the system described herein for screening of proteins.

FIG. 21 depicts an example of a colony plate having colonies picked according to the methods described herein.

FIG. 22 shows an example of how the models described herein extrapolates to all variants with different numbers of mutations further away from the wild type, compared to an industry standard.

FIG. 23 depicts an example of 384 well plate with cell pellets that contain enzymes expressed according to the methods described herein.

FIG. 24 depicts an example of seed culture optical densities (ODs) and the resulting main ODs at various shake speeds and well volumes for seed growth.

FIG. 25 depicts an example of processing of images for quantification of cellular proliferation as described herein.

FIG. 26 depicts an example of quantification of pellet areas and brightness for determination of cellular proliferation as described herein.

FIG. 27 depicts an example of a correlation between the ODs measured according to the imaging methods described herein and ODs measured with a spectrophotometer.

FIG. 28 depicts an example of a correlation between cell quantity (pellet OD) and product concentration for a given enzyme.

FIG. 29 shows an example of the use of the methods described herein can be used to drive substantial improvement in the performance of an enzyme.

FIG. 30 shows an example of how a substrate mesh can be used with the methods described herein to engineer novel enzyme activity.

FIG. 31 depicts an exemplary electronic device in accordance with some embodiments.

### DETAILED DESCRIPTION

The following description sets forth examples of methods, parameters, and the like. It should be recognized, however, that such description is not intended as a limitation on the scope of the present disclosure but is instead provided as a description of embodiments.

### Engineering Protein Activity

Engineering protein activity is approached here as a search and optimization problem. There is a near infinite size of the enzyme design space (i.e., nearly infinitely many possible amino acid sequences) as well as multiple and sometimes contradictory objectives (including activity, selectivity, stability), therefore human design (i.e., rational design) is inadequate. The systems and methods described herein can use new computational algorithms (also referred to herein as “models”) to engineer protein activity. In some cases, the protein activity catalyzes a transformation that is not found in nature and has not been (and/or cannot be) designed by previous methods.

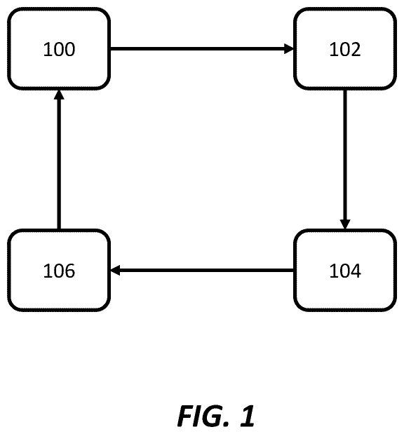

Without limitation, as seen in FIG. 1, the systems and methods can combine descriptive 100, predictive 102, and prescriptive 104 models. The descriptive models 100 can extract information from datasets (e.g., collected by a high-throughput robotic lab) and create labelled datasets (e.g., having protein sequence and activity data, on one or more substrates). The predictive models 102 can utilize the information from the labeled datasets to learn the complex mapping between the sequence of the protein and the various activities. The prescriptive models 104 can then interrogate these predictive models to more effectively search the near infinite design space and choose proteins that are expected to be optimal or near-optimal with respect to one or more design criteria. These chosen proteins can be expressed and screened against the substrate 106 (e.g., in a high-throughput manner) to generate additional data to feed the descriptive model 100. In summary, presented herein are robust and efficient algorithms for engineering of protein activity. These algorithms can also utilize transfer learning, thereby improving (exponentially) as the algorithms are used in successive protein design projects.

The systems and methods described herein can shorten the development time for proteins with novel activities and/or improved binding. In an aspect, the method can include proposing a plurality of candidate proteins using a prescriptive model that comprises a multi-objective search and optimization algorithm. For each of the plurality of candidate proteins, the method can further include predicting an enzymatic activity on a substrate using a predictive model that combines a machine learning algorithm and a physics-based simulation. At least some of the candidate proteins can be selected for expression (i.e., physical production of the enzyme). The activity on the substrate can be measured for the expressed candidate proteins.

Using the descriptive models described herein, the measured activity can be represented in a digital format that is compatible with the prescriptive and descriptive models. This dataset (including and/or derived from the measured activity) can be used to propose another plurality of candidate proteins using the prescriptive model. The measured activity can also be used to train or improve the prescriptive model and/or the machine learning algorithm of the predictive model.

The candidate proteins can be selected for expression based at least in part on the predicted activity on the substrate. In some cases, at least some of the proteins chosen for expression are chosen for their potential to improve the performance of the predictive and/or prescriptive models.

The selected candidate proteins can be expressed in any suitable host organism, including E. coli. The quantity of cells that are cultured can be measured (e.g., using image analysis of pelleted cells), i.e., for use as a proxy to an amount of the protein expressed (e.g., to normalize the measured activity on a per unit basis).

The activity of the expressed protein can be measured, e.g., in a high-throughput manner. The cells can be lysed to release the expressed proteins (e.g., using pressure, sonication, or chemical reagents). In some cases, the expressed proteins are enriched, however activity can also be measured in the cell lysate.

Measurement of activity can include conducting an enzymatic reaction, i.e., by contacting the expressed proteins with the substrate (along with any suitable co-factors or energy sources). The concentration of the substrate and/or a reaction product can be measured to determine an activity, using any suitable analytical method (e.g., a form of mass spectroscopy). The particular choice of detection method can depend on the substrate or product being detected.

The methods described herein can be performed in a massively parallel fashion. Many candidate proteins can be evaluated and/or chosen. Furthermore, an activity for each candidate protein can be predicted and measured on a plurality of substrates. These substrates can constitute a “substrate matrix” that can include the desired substrate (i.e., a desired industrial feedstock for conversion into a product). Other members of the substrate matrix can include substrates that have substantially similar structures to the desired substrate, but differ in a variety of ways. In some cases, these differences form a continuum between the desired substrate and the desired product. Data generated on substrates that are structurally adjacent to the desired substrate can improve performance of the systems and methods for engineering activity on the desired substrate.

The systems and methods described herein can also “transfer learn”. Here, at least two substrates can have substantially dis-similar structures. That is, the systems and methods described herein can be applied to different protein design challenges and the data from those instances can be aggregated. The aggregated dataset can grow over time (in overall size, within an area of design space, and in diversity of design spaces explored) such that the system performance can improve by building on itself. In some cases, the system can make useful predictions for proteins and/or reactions which it has never seen and/or do not exist in nature.

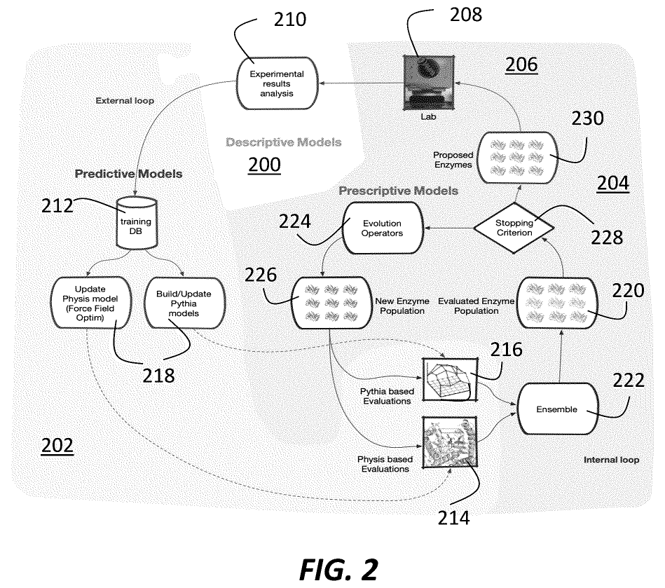

FIG. 2 shows the systems and methods described herein in additional detail. The system can comprise an external loop (following green arrows) and internal loop (following red arrows). Portions of the system are also color coded and include descriptive models 200 (yellow), predictive models 202 (blue), prescriptive models 204 (green), and a laboratory 206 (gray). The external loop can include a laboratory 208 for production and screening of proteins against substrates. The laboratory can be high-throughput (e.g., expressing thousands, tens of thousands, hundreds of thousands, or more proteins per day). The “laboratory” can also include data gleaned from various databases of activities measured by others.

Laboratory data can be messy (i.e., having experimental error, comprising a wide variety of proteins, reaction conditions, substrates, products) so the data can be pre-processed 210, e.g., using the descriptive models 200 described herein. The processed data can be added to a training database 212 that can be used to build or update (i.e., train) the predictive model. in some cases, the predictive model can have a physics-based simulation/model (“physis”) 214 and a machine learning algorithm (“pythia”) 216. Generation of data in a laboratory is also expensive and impractical for the exploration of any extensive proportion of protein design space. Therefore, this is done relatively less frequently and for relatively fewer proteins (external loop) compared with the number of iterations and proteins screened per iteration in silico (internal loop). The computational methods described herein can guide efficient use of laboratory resources.

Continuing with FIG. 2, an internal loop can be used iteratively many times for each use of the external loop. The activities of the candidate proteins can be predicted using one or more predictive models 202. The predictive models can be trained 218 on experimental data on the external loop and also used on the internal loop in conjunction with the prescriptive model 204. The predictive model can predict an activity of each candidate protein (along with other attributes such as a folded structure, stability, etc.) and add the result to a population of evaluated proteins 220. The physics-based simulation 214 and the machine learning algorithm 216 can combine and/or coordinate their outputs to arrive at a prediction through, e.g., an ensemble operation 222.

Ensemble models (also referred to as meta-algorithms) are a machine learning approach that combines multiple models in the prediction process. Those models can be referred to as base estimators. Ensemble models can have certain advantages over a single estimator, including lower variance, higher accuracy, and improved features noise and bias. The combination can be implemented by aggregating the output from each model with two objectives: reducing the model error and maintaining its generalization. Specific implementations include bagging (i.e., making training data available to an iterative process of learning where each model learns the error produced by the previous model using a slightly different subset of the training dataset), boosting (i.e., models are built on the top of models with lesser prediction capabilities), stacking (i.e., learning how to create a stronger model from the predictions of models having lesser prediction capabilities). The way to implement such aggregation can be achieved using some techniques.

In some cases, the predictive model 214, 216 is computationally expensive (i.e., takes a relatively long period of time to run and/or uses a relatively large number of processors). Thus, a prescriptive model 204 can be used, at least in part, to judiciously choose candidate proteins that would make best use of valuable computational resources of the predictive model. Evaluation operators 224 can be used to select an initial or subsequent group of candidate enzymes 226 for evaluation by the predictive model 214, 216. The prescriptive model can be based at least in part on a multi-objective search and optimization algorithm.

The prescriptive model 204 and predictive model 214, 216 (i.e., inner loop) can continue iteratively performing their functions until a stopping criterion 228 is reached. The stopping criterion can be based at least in part on the generation of a number of candidate proteins (e.g., proposed enzymes) that can be expressed and screened in the laboratory in a suitable period of time (e.g., 24-hours). This period of time can be substantially similar to the period of time it takes the algorithms to generate the candidate proteins such that the overall system is operating substantially continuously and the laboratory and computational resources are used effectively.

The result of the computational modeling can be a set of candidate proteins 230 that can either be used to generate additional data in the laboratory 208 and used to improve the models, or the process can be terminated (e.g., having a protein suitable or believed to be suitable for industrial or therapeutic use).

The systems and methods described herein can be used to engineer activity for any kind of protein such as a protein which catalyzes a chemical reaction (i.e., enzyme) or a protein which binds a substrate (e.g., antibody or fragment thereof). The protein can have any combination of natural or unnatural amino acids. The protein can be any size and/or have any number of amino acids, including a small number of amino acids (e.g., less than about 50), which are commonly referred to as peptides. The protein can be post-translationally modified in any suitable way, including glycosylation (including N-linked or O-linked), methylation, phosphorylation, acetylation, amidation, hydroxylation, ubiquitylation, or sulfation. The protein can have any suitable cofactor including an organic or inorganic cofactor (e.g., a metal ion, a metabolite, an iron-sulfur cluster, a vitamin, an energy carrier, an electron carrier, etc.). The methods described herein can also be used to engineer multi-protein or multi-subunit ensembles (i.e., comprising more than one polypeptide chain, which ensemble performs its desired activity while associating with its partners).

The substrate and product can be any suitable substances. Examples include a simple or complex substrate that forms an industrial feedstock for conversion into an industrial product such as a food product, a pharmaceutical product, a structural material, an adhesive, a fuel, etc. The product can be an intermediate product that is further transformed into the final product by another enzyme and/or other non-enzymatic steps. In some cases, a cascade of more than one enzyme is engineered using the methods described herein to form a production pathway from the substrate to the product (e.g., where a first enzyme makes an intermediate product for use by a second enzyme). The substrate can be another protein, a small molecule (e.g., glucose), or a polymer (e.g., cellulose). The substrate can be natural or man-made. In some cases, the enzyme protein acts on more than one substrate (at different times, or together). The enzyme can make or break covalent bonds, or can make or break no covalent bonds (e.g., only forming non-covalent bonds with a binding partner).

The systems and methods described herein can engineer any kind of activity. The activity can be an enzymatic activity or a binding activity on a substrate. Enzymatic activity can include a maximum reaction rate, a reaction rate at a certain reaction condition, a selectivity for a substrate or a product, a sensitivity to an inhibitory or activating factor, a reversibility of a reaction, or any combination thereof. The activity can also include the stability of the protein (e.g., in an organism or in an industrial environment), including its stability to degradation, its stability to fold or remain folded, and its stability in performing the desired reaction. Resistance to proteases can be a factor contributing to stability in a biological environment. Resistance to heat or cold can be a factor contributing to stability in an industrial environment. The engineered protein can also use/produce one or more enantiomers of a substrate/product preferentially over other enantiomers.

In some cases, the activity includes factors that are beyond the protein's ability to perform a reaction or bind a partner. For example, the methods described herein can be used to engineer or improve the cost or manufacturability of a protein (e.g., reduce the size of the protein). The protein can also be engineered to increase or decrease other factors included herein as “activity” (e.g., its immunogenicity or toxicity). Also included herein as “activity” is the patentability and/or freedom to operate (i.e., lack of infringement of existing patents) of the protein. In some cases, patent criteria can be built into the systems and methods to arrive at engineered proteins that are suitable for patenting and/or use in light of existing patents.

### Predictive Models

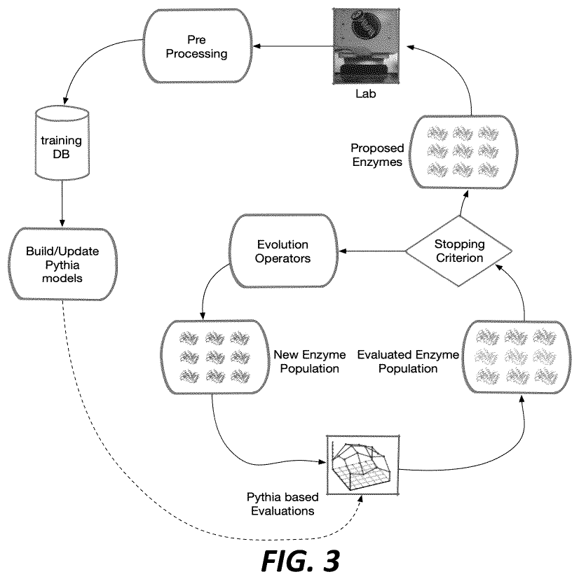

The predictive models described herein can be used to predict protein activity. The predictive model can have a trained machine learning model. In some cases, it also has a physics-based simulation model, however this is not required in all embodiments. FIG. 3 shows an example of the systems and methods described herein using a trained machine learning model without a physics-based simulation model.

In an aspect, provided herein is a method for evaluating a candidate protein. The method can include, by one or more computing devices, obtaining an amino acid sequence associated with the candidate protein. The amino acid sequence can be obtained from the prescriptive model. The amino acid sequences associated a plurality of candidate proteins can be obtained using a prescriptive model and input into the predictive model (i.e., the predictive model can be run in serial or in parallel for many candidate proteins). In some cases, distributed computation can be used to perform the methods described herein.

In some cases, the predictive model predicts protein activity only from the amino acid sequence of the protein. In other instances, information in addition to amino acid sequence is input into the predictive model. This can include the structure of the substrate (e.g., encoded in a form that can be used by the predictive model), structure of the protein, or any physiochemical property.

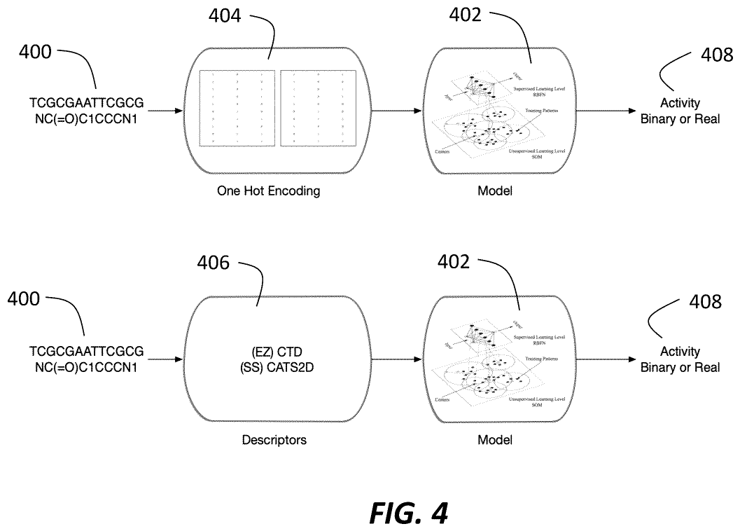

The method can include inputting the amino acid sequence into the predictive model to obtain a predicted activity on a substrate. As seen in FIG. 4, the information 400 can be input into the model 402 by one hot encoding 404 and/or by descriptors 406. Here, the information is a DNA sequence (top) and a coded structure for a substrate (bottom). The model can produce an activity for the protein. The activity 408 can be binary (i.e., an indication of whether the protein is expected to have any appreciable activity or not). The activity can also be quantitative (i.e., having not just a binary indication of activity, but an expected level or quantity of activity). Prediction of continuous activity levels instead of a binary classification (i.e., active or not) can allow design of enzymes with even higher activity, as the level of activity is additional information.

The predictive model comprises a trained machine learning model and optionally a physics-based simulation model. The physics-based model can be configured to provide features for the machine learning model of the predictive model. The features can be a set of physicochemical attributes that are assigned per amino acid residue or per atom, along with their corresponding structure-based vicinity graph. A structure-based vicinity graph is a coded version of a folded structure of the candidate protein. The features can be a folded structure for the candidate protein, a location at which the substrate is expected to dock with the candidate protein, expected vibrations of the substrate around a docked location of the candidate protein, an expected enzymatic reaction between the substrate and the candidate protein, or any combination thereof.

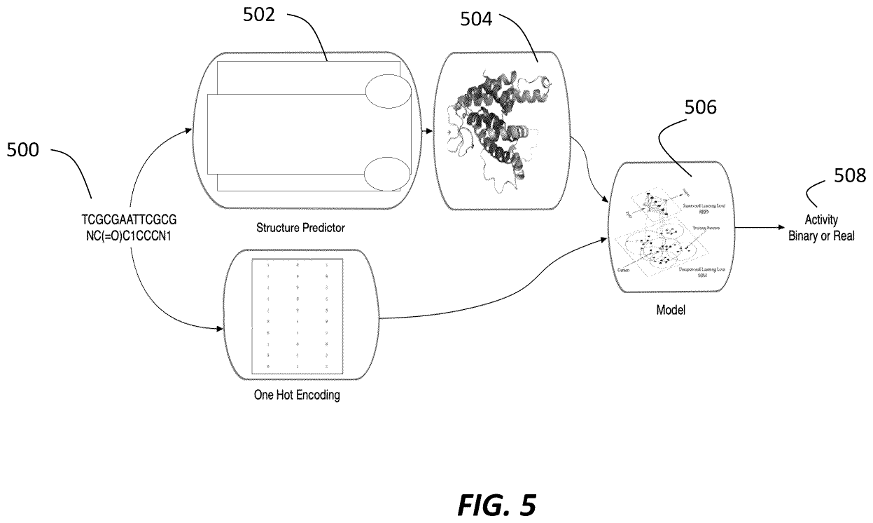

FIG. 5 shows that the amino acid sequence 500 and optionally any other information can be input into to a physics-based simulation model 502 (here, a structure predictor to yield a predicted folded structure 504 for the protein). The physics-based simulation model can yield a predicted activity or, as shown here, an intermediate input 504 that the trained machine learning module 506 can use at least in part to predict activity 508.

Incorporating a prediction of the folded structure of the protein into the method can enable extrapolation to more distant and differentiated enzyme designs and reactions from those having known activity. Models having a protein structure are explainable (e.g., allowing for supplement of rational design into the process, i.e., by humans) and are a more accurate representation of reality due to the application of physics constraints. In some cases, the methods described herein can incorporate an existing protein structure prediction algorithm, such as described in U.S. Patent Application Pub. No. 2021/0398606, which is incorporated herein by reference in its entirety. Briefly, such a method can include obtaining an initial embedding and initial values of structure parameters for each amino acid in the amino acid sequence, where the structure parameters for each amino acid comprise location parameters that specify a predicted three-dimensional spatial location of the amino acid in the structure of the protein. The method can further include processing a network input comprising the initial embedding and the initial values of the structure parameters for each amino acid in the amino acid sequence using a folding neural network to generate a network output comprising final values of the structure parameters for each amino acid in the amino acid sequence.

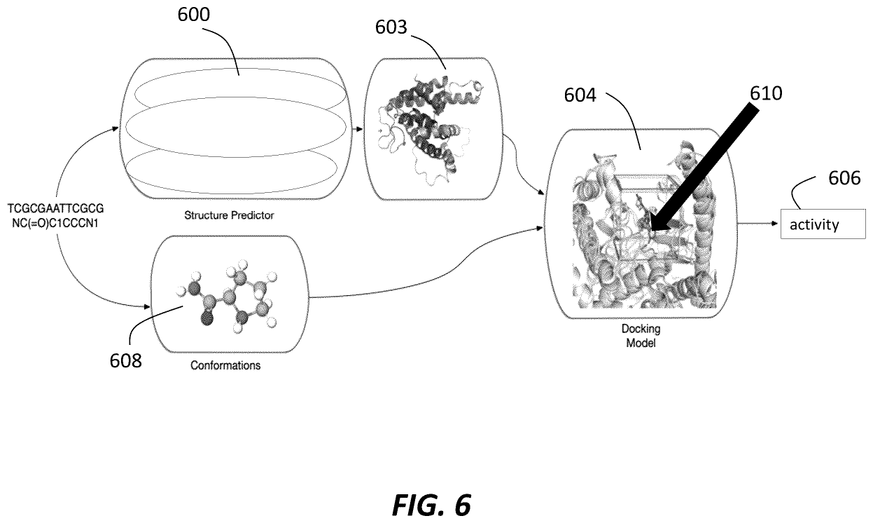

The physics-based model is configured to compute enzyme and substrate relative positions. In some cases, the predictive model can use more than one physics-based simulation in combination. For example, FIG. 6 shows a first physics-based simulation 600 (structure predictor) predicting a protein structure 602 that is used by a second physics-based simulation 604 (docking model) to predict the location and activity 606 for a molecule 608 in the folded protein 610. In some cases, the binding energy is the activity to be predicted (e.g., in the case of engineering a binding protein). In some instances, the binding energy is further used (e.g., by a trained machine learning algorithm) to predict an enzymatic activity. The molecule used in the docking model can be the substrate or an expected transition state (e.g., conformed or strained version of the substrate) in the conversion of the substrate to the product.

The physics-based model can comprise a molecular docking algorithm, a molecular dynamics algorithm, a quantum molecular dynamics algorithm, or any combination thereof. The molecular docking algorithm can be configured to find a likely location for the substrate to dock with the candidate protein. The molecular dynamics algorithm can be configured to predict vibrations of the substrate around a docked location of the candidate protein. The quantum molecular dynamics algorithm can be configured to simulate an enzymatic reaction between the substrate and the candidate protein.

The physics-based model can be configured to provide training patterns for the machine learning model of the predictive model. While metamodeling can be a simpler, faster, and/or cheaper, a ML model can be trained on a physics-based model's simulation results (i.e., use the physics-based output as its training patterns). This can over time (during the prescriptive algorithm's iterations) replace slow and expensive physics-based simulations with the aforementioned fast (cheap) model. In some cases, one keeps a database of all physics simulations. Once the database reaches a satisfactory number of entries, all candidate solutions have their objectives evaluated first using the cheap model (trained on the aforementioned database) and only the most promising ones are promoted to be evaluated by the more accurate and expensive physics models. The new physics-based simulation data are added into the database and the loop continues, while the cheap models become more and more accurate as they are trained on more and more data.

The physics-based model can constrain the machine learning model of the predictive model to a physical solution space. Here, the physics-based models are used to provide additional features to an ML-based predictive model tasked with predicting enzyme activity. Without limitation, these features can be inter-atomic distances between the substrate and binding pocket (or catalytic residues), binding energies, binding pocket accessibility, protein stability, and structure dynamics.

The machine learning model can predict the protein activity. In some cases, the machine learning model can be configured to predict a folded structure for the candidate protein (e.g., based on the amino acid sequence of the candidate protein). In some instances, the folded structure is predicted at least in part by a physics-based simulation. The folded structure can be used in the physics-based model and/or the machine learning model.

The systems and methods described herein can use any suitable machine learning model including but not limited to a convolutional neural network (CNN), a random forest (RF), a multilayer perceptron (MLP), a radial basis function network (RBFN), a graph neural network (GNN), or any combination thereof. The machine learning algorithm can have a supervised learning level and an unsupervised learning level.

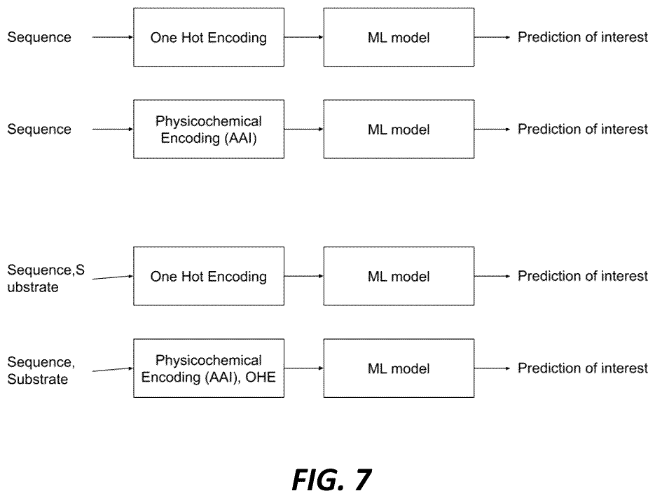

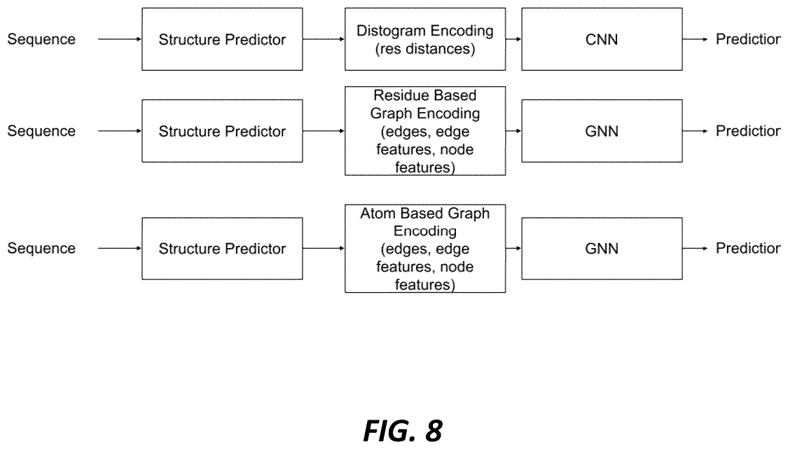

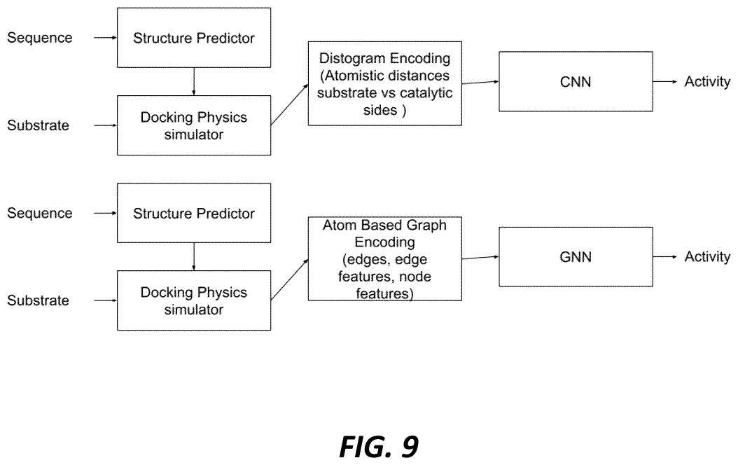

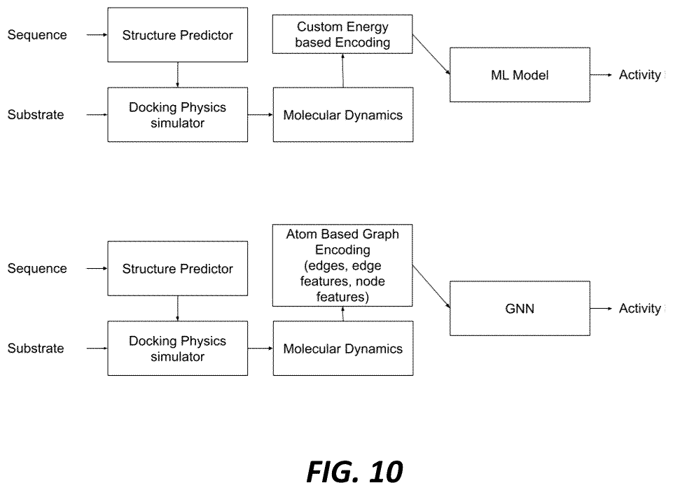

FIGS. 7-10 show various embodiments of the predictive models described herein. As used herein, OHE refers to one hot encoding and AAI refers to physiochemical properties (listed per amino acid).

The predictive model can learn from (i.e., be trained against) measured laboratory data. The predictive model can be configured to receive a measured activity for the candidate protein (i.e., through a training data set). The activity can be measured by expressing the candidate protein and exposing the expressed protein to the substrate, then the predictive model can be configured to be trained using the measured activity.

The machine learning model can be configured to be trained using a combination of stochastic and deterministic optimization methods. The training can increase accuracy of the predictive model for predicting activity on the substrate. The accuracy can be improved for sequences that are relatively far from any protein having a measured activity. For example, the increased accuracy of prediction can be achieved for a candidate protein having at least 3, at least 4, at least 5, at least 7, at least 10, at least 15, at least 20, at least 30, or at least 50 amino acid differences from any protein having a known activity on the substrate. The training can increase the accuracy by any amount including at least 10%, at least 20%, at least 50%, at least 100%, at least 5-fold, or at least 10-fold.

The predictive model can also be configured to be tuned using a measured activity. As used herein, “tuning” includes any performance improvement to the model (e.g., after an iteration of the external loop, generating additional laboratory data) that is less computationally demanding than fully re-training the model. The tuning can be performed by hot-starting from a version of the predictive model that has not been tuned using the measured activity. The tuning can take experimental error into account.

The method can include evaluating the candidate protein based on the predicted activity. The evaluation can comprise selecting one or more candidate proteins based at least partially on the predicted activity. The one or more candidate proteins can be selected for laboratory measurement of activity (e.g., to provide new training or tuning data) or selected for industrial, diagnostic, or therapeutic use.

### Prescriptive Models

It can be impractical to submit all desired candidate proteins to the predictive model for prediction of protein activity, e.g., because of constraints on computational resources. A driver of protein sequence diversity can also be needed to effectively explore protein sequence space. Furthermore, protein engineering is a multi-dimensional problem where “best” can be in relation to multiple, sometimes contradictory goals (e.g., reaction rate vs. selectivity). The prescriptive models described herein can achieve one or more of these needs.

In an aspect, provided herein is a method for identifying one or more candidate proteins. The method can comprise, by one or more computing devices, obtaining a plurality of candidate proteins using a prescriptive model comprising a multi-objective search and optimization algorithm. The method can further include inputting each candidate protein (e.g., inputting its amino acid sequence) of the plurality of candidate proteins into a predictive model to obtain a predicted activity on a substrate. One can select one or more candidate proteins from the plurality of candidate proteins at least partially based on the predicted activities.

The prescriptive model can constrain the number of candidate protein sequences submitted for analysis to the predictive model (i.e., the plurality of candidate proteins obtained using the prescriptive model is a sub-set of all of the proteins considered by and/or available for consideration by the prescriptive model). Furthermore, an activity on the substrate has typically not previously been measured for the candidate proteins obtained from the prescriptive model.

The candidate protein can be obtained from the prescriptive model based at least in part on its expected activity on the substrate. That is, the prescriptive model can make an initial prediction of protein activity, which is further verified and/or refined using the predictive model (i.e., which can be more accurate, but also more computationally expensive). In some cases, the models exchange information in both directions (e.g., prescriptive model uses the predictive model to make its proposals).

The prescriptive model can generate diversity amongst the candidate proteins. For example, the prescriptive model can be calibrated to explore sequences sufficiently divergent from those having measured activities in order to escape local design maxima and accelerate improvement of activity, yet not stray so far from sequences having measured activities such that predictions are low quality and/or too many experimental resources are wasted on negative results. The amount of sequence divergence can be any suitable value. In some cases, a candidate protein obtained from the prescriptive model has at least 3, at least 4, at least 5, at least 7, at least 10, at least 20, or at least 50 amino acid differences from any protein having a known activity on the substrate.

The candidate protein obtained from the prescriptive model is expected to have an optimal or near-optimal enzymatic quality in relation to the multiple objectives. The multiple objectives can comprise enzymatic activity, selectivity, stability, toxicity, size, novelty, or any combination thereof. In some instances, the enzymatic quality is on or in proximity to a pareto frontier.

The prescriptive model can be based at least partially on a combination of stochastic and deterministic optimization methods. A mixture of stochastic and deterministic mathematical optimization methods can be specifically designed to address high dimensional multi-objective constraint optimization problems with complex objective function landscapes. The stochastic method can be configured to randomly vary an amino acid identity at one or more positions of the candidate protein sequence. The deterministic method can be configured to compute and select an amino acid identity for one or more positions of the candidate protein sequence.

The prescriptive model can comprise a meta-model-assisted evolutionary algorithm. The meta-modeling algorithm can be configured to predict an enzymatic activity for a candidate protein based at least in part on the amino acid sequence of the candidate protein. The meta-modeling algorithm does not typically use a structure or predicted structure of the candidate protein (e.g., if obtaining a predicted structure is computationally demanding).

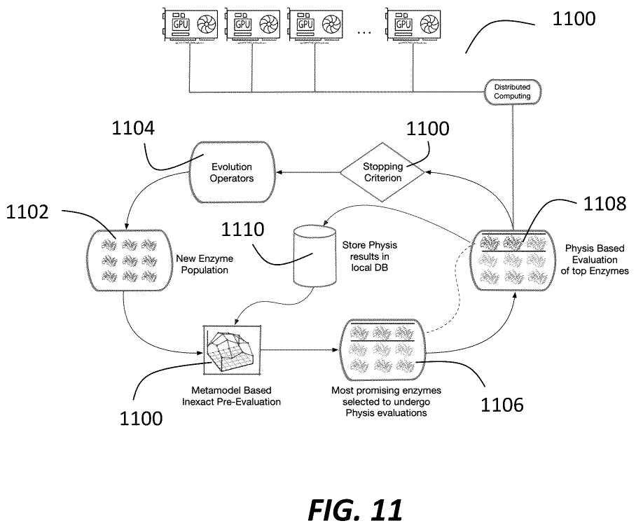

With reference to FIG. 11, a meta-model 1100 can be used in the method, at least in part, to take the initial population of candidate proteins 1102 produced by the evolution operators 1104 of the prescriptive model and choose the most promising proteins 1106 to undergo analysis by the predictive (physis) model. The results for the evaluated proteins 1108 can be stored to a (local) database 1110, which can be used to train and/or refine the metamodel 1100 to improve its activity predictions. The systems and methods (including the meta-model, the evolution operators, and/or the predictive model) can be run by distributed computing 1112, e.g., until a stopping criterion 1114 is reached. The red dotted line, are the results of evaluated proteins (using expensive physics and structure predictions). They are being stored into the database containing the metamodel's training dataset (i.e., as iterations proceed, the metamodel becomes a better predictor).

The meta-modeling algorithm can comprise a machine learning algorithm. The machine learning algorithm can include a random forest (RF), a multilayer perceptron (MLP), a radial basis function network (RBFN), a graph neural network (GNN), or any combination thereof.

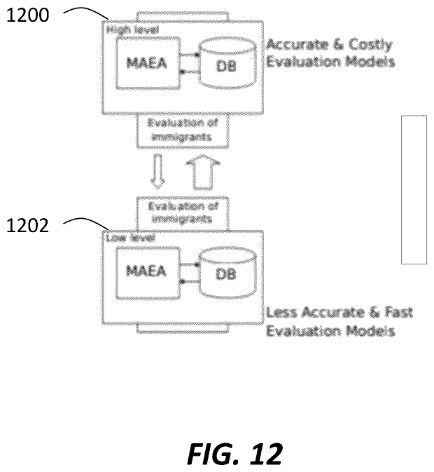

The prescriptive model can be further configured to use a hierarchical optimization algorithm. Referring to FIG. 12, the hierarchical optimization algorithm can combine a high-level model 1200 (i.e., that is relatively more accurate but more costly) with a low-level model 1202 (i.e., that is relatively less accurate but less costly, i.e., faster). Results (immigrants) can be shared between the lower level and the higher-level models. The prescriptive model can be configured to use levels (e.g., as higher and/or lower levels, in any configuration) of at least two of a docking model, a quantum-mechanical model, a machine learning model, and a molecular dynamics model.

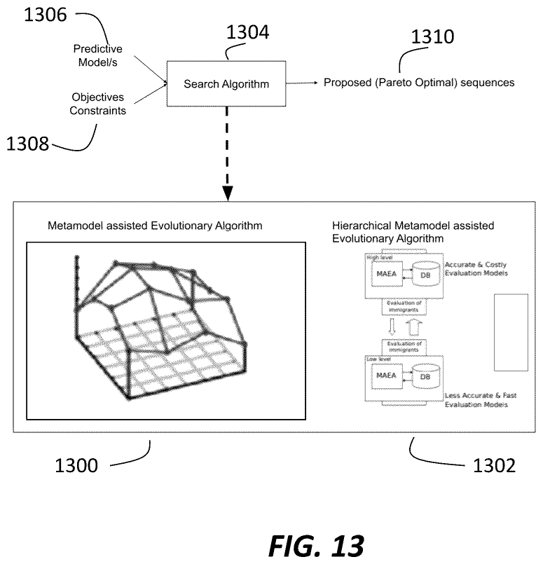

As seen in FIG. 13, a prescriptive model can have an metamodel assisted evolutionary algorithm 1300 and a hierarchical metamodel assisted evolutionary algorithm 1302. These algorithms (i.e., 1300, 1302) can be components of an overall search algorithm 1304 which takes predictive model (results) 1306 and objective constraints 1308 to propose (pareto optimal) sequences 1310 for expression and/or further analysis.

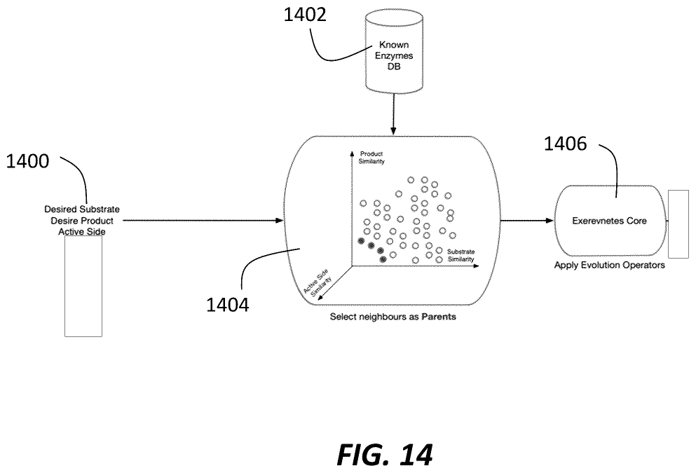

FIG. 14 shows another embodiment of a prescriptive model. Here, the desired substrate, product, and optionally active site 1400 are evaluated against a database 1402 of known (evaluated) proteins. Neighbors are selected 1404 as parents in an evolutionary algorithm (i.e., mutagenesis and/or shuffling) on the dimensions of similarity of the product, substrate, and/or active site. The evolution operators 1406 can be applied to yield candidate proteins. FIG. shows that the prescriptive model can use multiple approaches (operating in parallel and/or serial) to propose candidate protein 1500.

The one or more candidate proteins can be selected for measurement of activity. The activity can be measured by expressing the subset of candidate proteins and exposing each of the expressed proteins to the substrate. The prescriptive model can be further configured to receive measured activity for the candidate protein (e.g., and configured to be trained using the measured activity).

In another aspect, provided herein is a method for obtaining one or more candidate proteins comprising, by one or more computing devices, receiving an initial set of candidate proteins and obtaining the one or more candidate proteins by performing, based on the initial set of candidate proteins, one or more iterations of an evolutionary algorithm which utilizes problem-specific evolution operators. The problem-specific evolution operators are based at least in part on physics, protein structure, and/or amino acid sequence.

Each iteration can include evaluating a current set of candidate proteins and, based on the evaluation, updating the current set of candidate proteins. A subsequent set of initial candidate proteins can be generated and received by the evolutionary algorithm, based at least in part on the current set of candidate proteins. The prescriptive model can select one or more candidate proteins for measurement of activity.

The problem-specific evolution operators can comprise multiple sequence analysis (MSA). The evolutionary algorithm can include a multi-objective search and optimization algorithm.

The method can be based at least in part on amino acid sequences. E.g., the amino acid sequences of the initial set of candidate proteins can be received by the evolutionary algorithm and the amino acid sequences of the candidate proteins are obtained from the evolutionary algorithm.

The method can further comprise inputting the current set of candidate proteins into a predictive model, which predictive model predicts activity for each of the candidate proteins on a substrate. The predictive model can have a trained machine learning model and a physics-based simulation model.

The evolutionary algorithm can be assisted by a meta-modeling algorithm. The meta-modeling algorithm can include a machine learning algorithm. The meta-modeling algorithm can be configured to predict an activity for a candidate protein. The prediction of activity can be based at least in part on the amino acid sequence of the candidate protein.

In some instances, the meta-modeling algorithm does not use a structure or predicted structure of the candidate protein. The machine learning algorithm can include a random forest (RF), a multilayer perceptron (MLP), a radial basis function network (RBFN), a graph neural network (GNN), or any combination thereof. The meta-modeling algorithm can be further configured to receive measured activity for the candidate protein. The meta-modeling algorithm can be configured to be trained using the measured activity.

### Descriptive Models

The models comprise a set of signal processing, statistical and machine learning modules utilized to post-process (raw) experimental results and extract information from them (e.g., as required for use by the prescriptive and/or predictive models). The descriptive models can also interface with one or more analytics modules, enabling near real-time quality control and feedback to the lab (e.g., helping to support high-throughput protein production). In some instances, the descriptive model can efficiently and consistently analyze more than half a million samples per month. Use of broadband and multi-tiered screening techniques, paired with the descriptive models, can be a powerful tool for elucidating the space of possible chemical reactions.

### Protein Expression and Screening

The candidate proteins can be expressed in any suitable host organism, such as E. coli. In some cases, the protein is expressed in-vitro (e.g., using E. coli lysate). Any suitable detection method can be used to quantify a concentration of the substrate and/or product (e.g., a mass spectrometry method).

Existing systems for automated enzyme expression typically utilize 96 well plates for incubation and growth. Due to the low density of these plates, these processes require many plates per batch and thus are often incubated in manually operated incubators. In addition, many systems use large benchtop colony pickers that are selected for their high picking rates. These systems are not easily integrated and fall short in various automation features (APIs, image quality, etc.), preventing full walk-away ability. Finally, many existing systems require large liquid handling instruments for inoculating new plates. This produces a space constraint in most lab environments and manual operation of a single standalone instrument. Current labs are capable of processing many 96 well plates, but due to the lack of integration between modules, can require up to seven different systems. These drawbacks lead to discontinuous workflows that are expensive to run and maintain both from a consumables and human operation perspective, creating unfavorable scaling conditions.

In contrast, the systems and methods described herein can achieve high throughput enzyme expression using high density (e.g., 384 well plates). The present disclosure overcomes challenges for growing E. coli in 384 well plates caused by the geometry of the plate, which effectively reduces the available surface area and thus oxygenation. These challenges are overcome by novel growth conditions including culture volume, plate seal conditions, shake speeds and throw, and media selection. A suite of instruments integrated into the system described herein can enable the creation of thousands of new enzyme variants in a day. The density and volume of cultures that are grown on a plate can depend on the production organism (i.e., 96 well plates can be preferred for some hosts, e.g., that are not e-coli).

In particular, the systems described herein have one or more of the following features: (a) integrated hardware for automated colony imaging, picking, growth and expression, (b) integrated cell pelleting, harvesting and freezer storage, (c) high-density liquid transfer robots that do not require pipette tips, thus enabling continuous operation and cost reduction, (d) automated application of both breathable and non-breathable plate seals, (e) automated growth and expression in 384 well format, and (f) automated image-based quantification of cell density. These features provide a solution which allows for maximum walk-away time, minimized consumables costs (e.g., less than 10 cents/sample), and complete end-to-end automation in some cases.

In an aspect, described herein is a method for high-throughput automated expression of a plurality of proteins. The method can comprise providing a colony plate having colonies of cells dispersed thereon, where the cells are capable of expressing one of a plurality of proteins; robotically inoculating a plurality of cultures from the colonies of cells, where the cultures are arrayed in wells of a culture plate; incubating the culture plate at conditions suitable for growing the cells in the cultures; and robotically harvesting the cells from the cultures to provide cell pellets. In some cases, a colony of a culture plate can have cells that are capable of co-expressing more than one protein (e.g., as a strategy that increases the number of enzyme variants that can be screened for activity, especially in cases where many variants are not active on the substrate).

The method can be high-throughput. In some cases, a single person is capable of incubating about 2,000, about 4,000, about 10,000, about 20,000, about 30,000, about 40,000, about 50,000, about 75,000, about 100,000 or more cultures in a day. In some instances, a single person is capable of incubating at least about 2,000, at least about 4,000, at least about 10,000, at least about 20,000, at least about 30,000, at least about 40,000, at least about 50,000, at least about 75,000, or at least about 100,000 cultures in a day. The number of cultures performed (e.g., in 384 well plates) can correspond to the number of distinct proteins produced (i.e., if each culture expresses a unique protein).

The method can be automated. For example, the person does not physically contact the culture plate, the colony plate, or a fluid handling device between when the colony plate is provided and the cells are harvested. Integrated modules utilizing robotics can transfer materials between operations as described herein.

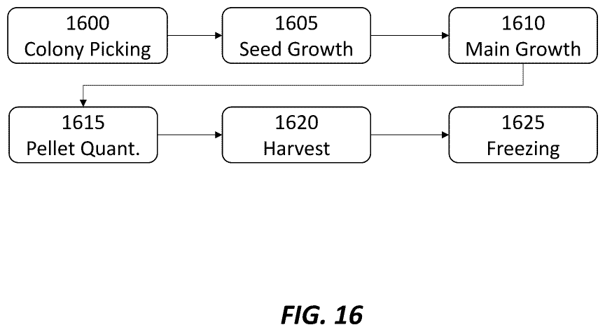

With reference to FIG. 16, the method described herein can include colony picking 1600, seed growth 1605, main growth 1610, pellet quantification 1615, harvest 1620 and freezing of cell pellets 1625. In some cases, a single round of liquid culture can be adequate (i.e., a seed growth culture is not needed prior to a main growth culture). Nevertheless, in some embodiments, the method further comprises, following incubating the culture plate, robotically inoculating a plurality of second cultures from the incubated first cultures, where the second cultures are arrayed in wells of a second culture plate; and incubating the second culture plate at conditions suitable for growing the cells in the second cultures.

The cells can be harvested from their growth medium using centrifugation. In some cases, the method further comprises freezing the harvested cells. The frozen cells can be thawed or otherwise processed for downstream uses at a later date (e.g., for enzymatic screening as described herein). The cells can be frozen at any suitable temperature (e.g., −20° C., −80° C.) for any period of time. In some cases, the method can involve lysing the harvested cells (e.g., by use of pressure or lysing reagents), for example, to release the expressed protein for further analysis. In some cases, the expressed protein can be enriched from the lysed cells (e.g., by centrifugation or affinity capture). The enriched and/or liberated protein can be reconstituted in a suitable solution, re-folded, post-translationally modified, or subjected to any desired treatment. The cells can be harvested in any suitable buffer (e.g., an ammonium carbonate buffer).

In some embodiments, the method further comprises optically quantifying the sizes of the cell pellets as described herein, thereby providing a measurement of an amount of the cells grown in the culture (e.g., as an indirect measure of the amount of protein expressed). In some cases, an amount of expressed protein can be measured more directly. The amount of expressed protein can be used to normalize later measured enzymatic activities on a per mass or per mol of enzyme basis.

DNA can also be liberated or enriched from the cultured cells. This DNA can include the DNA that encoded for the desired protein. In some cases, the encoding region of the liberated DNA can be sequenced with any suitable DNA sequencing method (e.g., to determine the sequence identity of the protein expressed in each well of the culture plate).

The method can be performed to reduce reagents or other consumables expended in the process. In some embodiments, the method is performed without disposable pipette tips. For example, the cultures can be robotically inoculated using sterilizable and reusable pins.

The culture plate is incubated at conditions suitable for expressing the protein in the cell. This can include any suitable growth temperature, humidity, atmospheric concentration, media composition, and the like. The conditions can include (or exclude) factors that result in expression of the protein from an inducible promoter. In some cases, the culture plate is agitated at a certain number of revolutions per minute (RPM), e.g., to promote gas exchange. The rate of agitation can be about 500, about 750, or about 1,000 RPM (see, e.g., Example 5).

A system is provided herein for high-throughput automated expression of a plurality of proteins. The system can comprise: a reagent dispenser configured to dispense media into wells of a culture plate; a colony picker configured to pick colonies of cells from a colony plate and inoculate a plurality of cultures in wells of the culture plate; an incubator configured to incubate the culture plate at conditions suitable for growing the cells; a harvester configured to enrich the cells from the culture plate to provide cell pellets; and a plate transfer module configured to move the culture plate between the reagent dispenser, the colony picker, the incubator, and the centrifuge. In some cases, the system is capable of incubating at least 2,000 cultures in a day. A plate sealer can be configured to seal the wells of the culture plate from an atmosphere and a plate peeler can be configured to remove a seal applied by the plate sealer.

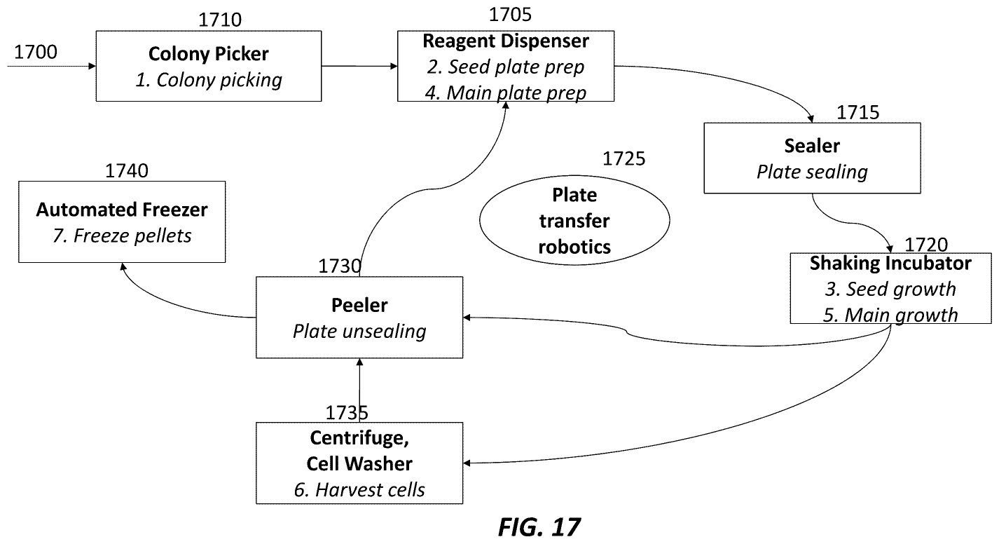

An example of the system for high-throughput automated protein expression is shown in FIG. 17. Here, a plurality of colony plates are provided 1700. A reagent dispenser 1705 can be configured to dispense media into wells of a culture plate. This media can be a liquid media suitable for culturing the transformed protein production host cells (e.g., including suitable nutrients, growth factors, and selection antibiotics). The colony plates can be loaded into a colony picker 1710 which is configured to pick colonies of cells from a colony plate and inoculate a plurality of cultures in wells of a culture plate. A sealer 1715 can apply a seal over the culture plate (e.g., to prevent contamination and/or excess loss of culture medium by evaporation). In some cases, the applied seal is permeable to gasses (e.g., O₂, CO₂) to facilitate respiration of the cultured organisms. The culture plate can then be incubated in a shaking incubator 1720. Plate transfer robotics 1725 can move the plate(s) between modules. In some cases, a single round of culture growth produces an adequate and/or consistent quantity of cells.

In some cases, a second round of culture growth is needed to produce adequate and/or consistent quantity of cells. Here, a peeler 1730 can remove the seal from the culture plate. The reagent dispenser 1705 can dispense growth media into wells of a second culture plate. An inoculum from the first culture plate can be transferred to the second culture plate. The sealer 1715 can seal the second culture plate, which can be incubated in the incubator 1720. Continued iterations or parallel productions of culture can be performed for any desired purpose.

Upon the completion of the final culture phase, the plate can be harvested 1735 to harvest (i.e., pellet) the cells. The harvester can include a centrifuge (e.g., centrifuge, rotovap). The centrifuge and reagent dispenser can be used to discard spent medium and/or wash the pelleted cells. The cells can be subjected to further processing at any stage (e.g., before or after freezing). In some cases, the system further comprises a freezer 1740.

The system can be automated. For example, a person may not physically contact the culture plate or the colony plate other than to provide empty consumable plates to the system and/or remove waste plates from the system.

The expression system described herein can be capable of automating a 3-day expression process from colony picking to frozen cell pellets, with minimal hands-on time and without costly pipette tips normally required by similar systems. In addition, the cell culture volumes, shake speeds and media have been selected to work with 384 well plates for increased throughput. The system can be capable of incubating any suitable number of plates simultaneously. In some cases, 32 plates are incubated simultaneously (yielding 12,288 enzymes).

### Cell Growth Quantification

Optical density (OD) is a common measurement used to monitor and quantify cell culture growth. OD is most commonly measured in microtiter plates whereby a sample of microbial cell culture is removed from the incubating vessel, diluted, and measured for optical density (e.g., at 600 nm). Based on the cell type that is grown, a correlation between OD and cell number or cell mass can be established. This process requires consumables to collect and dilute the samples appropriately, and also use of an expensive spectrophotometer.

However, OD is ill-suited for quantification of cell growth in the low-volume, high-throughput system described herein. First, OD sampling and dilution adds an excessive number of materials and steps to the process (e.g., including additional plate for OD measurement). Second, sampling from low volumes consumes an unacceptable fraction of the total culture volume. Finally, the desired measurement is not a growth time course, but only the quantity of cells at the completion of the culture (i.e., at harvest, e.g., to normalize measured enzyme activities).

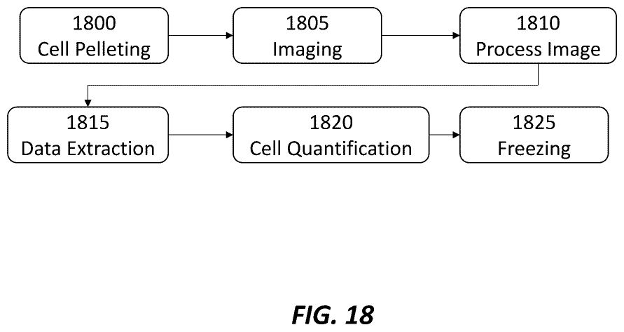

Thus, with reference to FIG. 18, provided herein is a method for quantifying an amount of cellular proliferation. The method can comprise centrifuging a plate containing an array of cell cultures to provide an array of cell pellets in a solution 1800. The solution (e.g., spent media) can optionally be removed from the plate. The plate can be imaged 1805 to provide an image. This can be performed using a camera or a (flat-bed document) scanner. The images can be processed 1810 (e.g., to standardize them). Data associated with the cell pellets can be detected 1815 from the image. The data can include a location of each cell pellet, a size of each pellet, and/or a brightness of each pellet. The data associated with the cell pellets can be used to quantify 1820 a mass or number of cells in each cell pellet. The quantified cell pellets can be frozen 1825 for future use. The method can be capable of quantifying a mass or number of cells in at least 96 or at least 384 cell pellets from a single image.

A potential advantage of the method for cell quantification described herein is that there is no sampling (i.e., substantially all of the cells in the cell cultures are in the cell pellets). Another advantage includes more easily maintaining sterility of the mono-culture. In some embodiments, the method further comprises reconstituting the cell pellets and/or performing a subsequent procedure on substantially all of the cells.

The imaging can be performed with any suitable device (e.g., a camera). In some cases, a scanner can be modified to keep a background constant and/or to put the plate in a location on scanner. In some cases, the cell pellets are modified to over-express a fluorescent protein and the imaging is performed at an emission wavelength, but this is not required. The imaging can use any suitable wavelength(s) (e.g., the visible spectrum).

In some embodiments, the method further comprises processing the image prior to detecting data associated with the cell pellets, wherein said processing comprises at least one of cropping the image, aligning the image, or thresholding the image.

The pellet image can be pixelized with any suitable resolution. In some embodiments, a sample of about 50, about 100, about 200, about 300, about 600, about 900, about 1,500, about 2,000, about 5,000, about 10,000, or about 100,000 pixels are used to determine the mass or number of cells.

Brightness of the pixels can be used to quantify the size and/or density of the cell pellet. The individual pixels can be scored by brightness. In some embodiments, a plurality of pixels of the image are scored on a scale of brightness ranging from 0 to 255.

A trained machine learning algorithm can be used to convert the image (pixels and scores) into a quantity of cells. Following training, this can be done on an absolute basis rather than in reference to a standard. That is, quantifying a mass or number of cells in each cell pellet does not include comparing an image of a cell pellet with a reference image that was generated at substantially the same time as the image of the cell pellet to be quantified. The machine learning algorithm is trained on images acquired from cell pellets having a known mass or number of cells.

### Protein Screening

Some existing protein engineering systems involve the use of 96-well microtiter plates and colorimetric enzymatic assay endpoints. These workflows are limited in their capacity, as they use large reaction volumes (e.g., 100-1,000 microliters) and are therefore unable to screen against many substrates at once. In contrast, the screening platform described herein lyses cell pellets to release active enzymes, aliquots them, and combines them with one or more substrates (e.g., a substrate mesh) to perform enzyme activity assays in high throughput. The system has sustained large-scale screening campaigns utilizing enzymatic assays at 7 microliter volumes in 1,536-well plates. The screening system can measure more than 30,000 enzyme reactions in a day while maintaining less than 10 cents consumable cost per enzyme reaction.

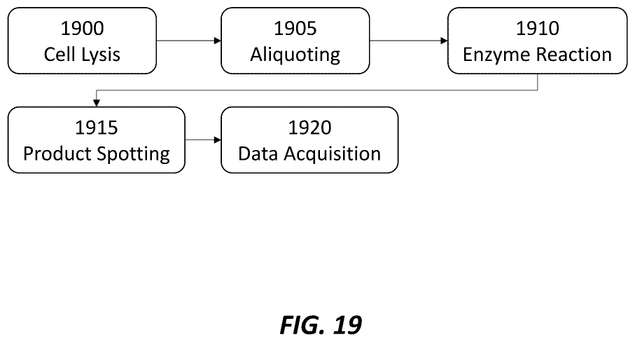

With reference to FIG. 19, provided herein is a method for high-throughput automated functional screening of proteins. The method can include providing a plurality of proteins, e.g., by releasing them from lysed cells 1900. The proteins can be aliquoted 1905, e.g., in order to make duplicate activity or binding measurements in order to have greater statistical certainty in the measured result. The proteins can also be aliquoted in order to measure activity against a panel of substrates or ligands. The method can then include performing enzymatic (or binding) reactions (including inhibition of enzyme activity) 1910 by robotically combining each of the plurality of proteins with a substrate on a reaction array and incubating the reaction array to produce a plurality of reaction products. The incubation can be at conditions that are suitable for the proteins to transform the substrate into a reaction product. The robotic transfer 1915 of the reaction products to a detection array (i.e., spotting) can prepare the reaction products for detection as described herein. In some cases, less than all of the reaction product is needed for a detection and multiple detections can be performed for each reaction, thereby giving greater statistical confidence in the measurement. Finally, the method can include detecting the reaction products 1920 on the detection array (i.e., data acquisition).

The method can be high-throughput. In some cases, a single person is capable of detecting at least about 10,000, at least about 20,000, at least about 50,000, at least about 100,000, at least about 200,000, at least about 500,000, at least about 1,000,000 reaction products in a day.

The method can be automated. In some embodiments, the person does not physically contact the reaction array, the detection array, or a fluid handling device between when the plurality of proteins are provided and the reaction products are detected.

The enzyme reaction can be performed at any suitable set of conditions, including multiple conditions. The reaction conditions can replicate the conditions that are expected to be used for industrial production of the product. The conditions (e.g., temperature, presence of a solvent) can be selected to test the stability of the enzyme at those conditions. In some cases, a concentration of the substrate can be varied, or the concentration of various inhibitors or promoters of the reaction can be varied. In some cases, the amount of protein analyzed is small. About 1, about 2, about 5, about 10 microliters (uL) of protein can be robotically combined with the substrate. The proteins are provided in cell lysates or enriched from cell lysates.

In some embodiments, the robotic combination and/or the robotic transfer are performed without disposable pipette tips, using sterilizable and reusable pins, or using an acoustic liquid handler. In some embodiments, the reaction array comprises at least about 1,536 positions. In some cases, positions of the reaction array have a volume of between about 5 and about 7 microliters (uL).

The method can use any suitable detection method, such as mass spectrometry. In various embodiments, the detecting can be performed using laser desorption and ionization (LDI), including matrix-assisted desorption and ionization (MALDI) or surface-assisted desorption and ionization (SALDI). The detecting can be performed using liquid chromatography mass spectrometry (LCMS), gas chromatography mass spectrometry (GCMS). The detection method can also be performed without mass spectrometry detection (e.g., gas or liquid chromatography). In some cases, the detecting is performed using fluorescence or absorbance detection.

Screening thousands of reactions a day requires management of false positives and false negatives inference errors, e.g., in search the sequence and fitness landscape. A tiered screening approach can be used where an ultra-fast and high throughput first tier casts a wide net and is followed by higher resolution/lower throughput second (and third) tier screen that can validate hits identified in the first screen, ultimately reducing false positives. The goal is to maximize screening targets, minimize cost per experiment, and minimize inference errors.

The method can use multiple detection methods. In some embodiments, plurality of proteins are robotically combined with a second substrate on a second reaction array, the second reaction array is incubated to produce a plurality of second reaction products, and the second reaction products are detected on a second detection array.

The amount of reaction product sampled for detection can depend on the detection method. In some cases, there is a 100-fold or more reduction in volume from the enzyme reaction to the (e.g., label-free) detection methods. In some cases, about 10, about 20, about 50, about 75, about 100, about 200, about 500, or about 1000 nanoliters (nL) of reaction mix is used for detection. With small volumes, this can allow at least 20, at least 40, at least 100 replicates. In some cases, 64 replicates are used for acquisition, which can be valuable during second tier confirmations.

The detection array can be used and modified in a manner suitable for use by the detection device. In some cases (e.g., mass spectrometry) the reaction products are dried on the detection array. In some embodiments, the detection array comprises at least about 6,144 positions.

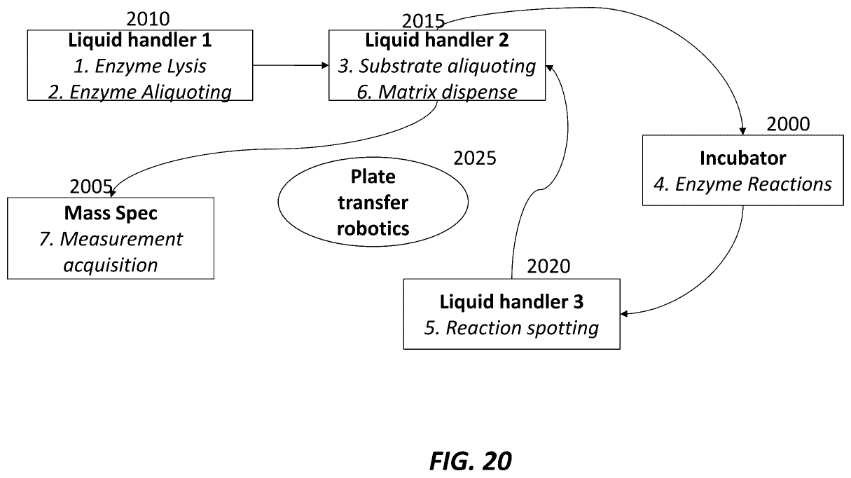

In another aspect, provided herein is a system for high-throughput automated functional screening of proteins. With reference to FIG. 20, the system can comprise an incubator 2000 configured to incubate a plurality of proteins with a substrate on a reaction array to produce a plurality of reaction products and a detection module 2005 (e.g., mass spec) configured to detect the reaction products on a detection array. One or more liquid handlers 2010, 2015, 2020 can be configured to lyse cells containing the protein, aliquot the protein onto the reaction array, aliquot the substrate onto the reaction array, and/or transfer some of the reaction products from the reaction array to the detection array. A plate transfer module 2025 can be configured to move the reaction array and/or the detection array between the incubator, the detection module, and the one or more liquid handlers. The system can be high-throughput and automated.

In another aspect, provided herein is a method for high-throughput automated expression and functional screening of proteins. The method can comprise: providing a colony plate having colonies of cells dispersed thereon, wherein the cells are capable of expressing one of a plurality of proteins; robotically inoculating a plurality of cultures from the colonies of cells, wherein the cultures are arrayed in wells of a culture plate; incubating the culture plate at conditions suitable for growing the cells in the cultures; robotically harvesting the cells from the cultures to provide cell pellets; imaging the cell pellets and using the image and a trained machine learning algorithm to quantify a mass or quantity of cells in the cell pellets; robotically combining each of the plurality of proteins from the cell pellets with a substrate in a reaction array; incubating the reaction array to produce a plurality of reaction products, which incubation is at conditions that are suitable for the proteins to transform the substrate into a reaction product; robotically transferring the reaction products to a detection array; and detecting the reaction products on the detection array. In some cases, a single person is capable of detecting at least 10,000 reaction products in a day.

In another aspect, provided herein is a system for high-throughput automated functional screening of proteins, the system comprising: a colony picker configured to pick colonies of cells from a colony plate and inoculate a plurality of cultures in wells of a culture plate; one or more incubators configured to incubate the culture plate at conditions suitable for growing the cells and expressing a protein in the cells, and incubate a plurality of proteins with a substrate on a reaction array to produce a plurality of reaction products; a harvestor configured to harvest the cells from the culture plate to provide cell pellets; a detection module configured to detect the reaction products on a detection array; one or more liquid handlers configured to dispense media into wells of a culture plate, lyse cells containing the protein, aliquot the protein onto the reaction array, aliquot the substrate onto the reaction array, and/or transfer some of the reaction products from the reaction array to the detection array; and a plate transfer module configured to move the culture plate, the reaction array and/or the detection array between the one or more liquid handlers, the colony picker, the one or more incubators, the centrifuge and the detection module.

### Molecular Biology, Information Management, and Control Systems

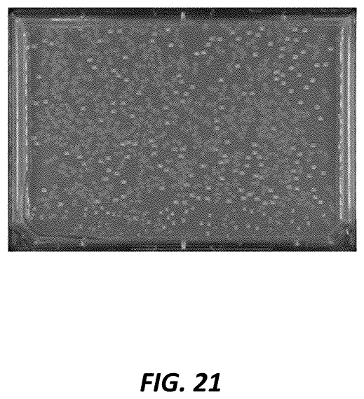

The systems and methods described herein can start from colony plates. The colony plates can have discrete colonies of a microbial production host (e.g., e-coli, yeast, mammalian cells) which are expressing or are capable of expressing a plurality of proteins (e.g., variants of a protein library or group of un-related proteins) such as shown in FIG. 21. Here, the selected (picked) colonies are marked in red. Methods for producing a colony plate are well known in the art and can include methods for synthesizing the gene of interest that encodes for the desired protein, cloning that gene behind a suitable promoter (e.g., constitutive or inducible promoters) in a suitable vector (e.g., a plasmid), transforming the microbial host (e.g., by electroporation or using chemical means), and plating the transformed hosts on a suitable medium for growth of colonies (e.g., on complex or minimal media, optionally including an antibiotic for the selection of transformed cells). The medium on the colony plate can be a solid medium.

The proteins being expressed and screened can be any suitable protein. In some embodiments, the proteins are enzyme variants. The variants (and diversity thereof) can be created by any suitable method including random mutagenesis, site-saturation mutagenesis, DNA shuffling (combinatorial mutagenesis), rational or semi-rational design.

For each production plate that goes through data collection, there can be several different types of process controls that allow monitoring performance. A spectral analysis pipeline can analyze mass spectra after they are acquired, identifying peaks related to the ionization of various compounds present in the sample. In some cases, this peak calling functionality is distinct from a detections pipeline, which can use a statistical measure of peak detection. By tracking samples and controls across wells and plates, the method described herein allows one to rapidly and consistently analyze the measurements and troubleshoot processes ranging from liquid dispensing to instrument calibration issues.

The visualization of product-to-substrate ratios (PSR) calculated for spectra on a measurement plate can be used in the method described herein. PSR can be an important metric for quantifying enzyme activity, as it can be sensitive to both the chemical generation of product compounds and the depletion of substrate. A heat map can show regions with different intensities of PSR, providing one with a quick verification that the expected samples have been correctly deposited in columns as intended in the experimental design. The methods described herein can also use other ratios, such as product to total ratio (PTR).

In some embodiments, the system further comprises a scheduler configured to coordinate the components of the system. In some cases, the system further comprises a laboratory information management system (LIMS) configured to record and/or provide data associated with the system.

The LIMS (Laboratory Information Management System) described herein can satisfy some of the unique requirements and challenges of a high-throughput automated laboratory. The LIMS can provide an easy-to-use user interface. It can efficiently manage the complexity of capturing and tracking millions, billions, or more data points from the laboratory and present the data to the end user in an organized and intuitive fashion. In addition, all of the LIMS functionalities can be exposed in APIs, making the LIMS solution primed to support fully automated lab systems.

The methods described herein can use detailed sample tracking. The laboratory processes, such as colony picking, cell lysis, DNA sequencing, liquid transfer, and reaction incubation, involve many complex steps. All of the data coming from each step can be linked so that the system can capture a complete picture from sample contents to the reaction screening results. The vast quantity of data is tied together accurately in an efficient and highly scalable manner. The data structures and traversal logic are designed so that many levels of hierarchical data, and details thereof, can be brought together quickly and efficiently.

The system can use procedure-centered workflow modeling. One of the components in laboratory operations is building LIMS features that can model, track, and pre-validate physical lab procedures. Accurate modeling helps ensure the accuracy and validity of the manufacturing record and provides an additional layer of protection against errors like plate swaps or incorrect loading of automation systems.

The LIMS has developed a very flexible workflow solution that enables lab processes to be described and tracked. Instead of modeling each process as a “one-off” solution that requires a new LIMS release, the workflow management can use a component-style architecture to represent common workflow patterns, and a mechanism to describe the workflows using a procedure template format that largely consists of plain configuration metadata.

All components can be explicitly versioned, making it possible to run development procedures in parallel with production procedures. Allowing for easy parallelism means procedures can easily evolve over time, typically without depending on LIMS release.

The overall solution also gives one the capability to validate lab inputs and describe/enforce conditions that must be met before process steps can advance. Lab automation robots can then have an independent source of truth when operating. The module also tracks information that enables insight into the timing, sequencing, success, and re-attempt of operations as they occur in the lab, which is a valuable foundation for making ongoing improvements and scheduling optimization.

The LIMS tracks dozens of quality measures per well/sample. Many of these fall under the umbrella of DNA sequencing quality checks and quality measures pertaining to the screened results.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In this respect, it should be appreciated that one implementation of the embodiments of the present invention comprises at least one non-transitory computer-readable storage medium (e.g., a computer memory, a portable memory, a compact disk, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

### Automated Platform

In an aspect, provided herein is a method for high-throughput automated expression of a plurality of proteins. The method can include providing a colony plate having colonies of cells dispersed thereon, wherein the cells are capable of expressing one of a plurality of proteins. The method can further include robotically inoculating a plurality of cultures from the colonies of cells, wherein the cultures are arrayed in wells of a culture plate. The method can further include incubating the culture plate at conditions suitable for growing the cells in the cultures. The method can further include robotically harvesting the cells from the cultures to provide cell pellets. A single person can be capable of incubating at least 2,000 cultures in a day.

In some embodiments, the person does not physically contact the culture plate, the colony plate, or a fluid handling device between when the colony plate is provided and the cells are harvested.

In some embodiments, the method further comprises, following incubating the culture plate: robotically inoculating a plurality of second cultures from the incubated first cultures, wherein the second cultures are arrayed in wells of a second culture plate; and incubating the second culture plate at conditions suitable for growing the cells in the second cultures.

In some embodiments, the method further comprises, optionally freezing the harvested cells; and lysing the harvested cells. In some embodiments, the harvested cells are frozen.

In some embodiments, the method further comprises, optically quantifying the sizes of the cell pellets, thereby providing a measurement of an amount of the cells.

In some embodiments, the culture plate is incubated at conditions suitable for expressing the protein in the cell. In some embodiments, the method is performed without disposable pipette tips. In some embodiments, the cultures are robotically inoculated using sterilizable and reusable pins.

In some embodiments, the cells are harvested in an ammonium carbonate buffer.

In some embodiments, the proteins are enzyme variants.

In another aspect, provided herein is a method for high-throughput automated functional screening of proteins. The method can include providing a plurality of proteins; robotically combining each of the plurality of proteins with a substrate on a reaction array, incubating the reaction array to produce a plurality of reaction products, which incubation is at conditions that are suitable for the proteins to transform the substrate into a reaction product; robotically transferring the reaction products to a detection array; and detecting the reaction products on the detection array. In some cases, a single person is capable of detecting at least 10,000 reaction products in a day.

In some embodiments, the person does not physically contact the reaction array, the detection array, or a fluid handling device between when the plurality of proteins are provided and the reaction products are detected.

In some embodiments, the plurality of proteins are robotically combined with a second substrate on a second reaction array, the second reaction array is incubated to produce a plurality of second reaction products, and the second reaction products are detected on a second detection array.

In some embodiments, the proteins are provided in cell lysates. In some embodiments, the proteins are enriched from cell lysates.

In some embodiments, the detecting is performed using mass spectrometry. In some embodiments, the detecting is performed using laser desorption and ionization (LDI).

In some embodiments, the detecting is performed using matrix-assisted desorption and ionization (MALDI). In some embodiments, the detecting is performed using surface-assisted desorption and ionization (SALDI). In some embodiments, the detecting is performed using liquid chromatography mass spectrometry (LCMS) or gas chromatography mass spectrometry (GCMS).

In some embodiments, the detecting is performed using fluorescence or absorbance detection. In some embodiments, less than about 20 uL of protein is robotically combined with the substrate.

In some embodiments, the robotic combination and/or the robotic transfer are performed without disposable pipette tips.

In some embodiments, the method is performed using sterilizable and reusable pins. In some embodiments, the method is performed using an acoustic liquid handler. In some embodiments, the reaction array comprises at least about 1,536 positions.

In some embodiments, positions of the reaction array have a volume of less than about 20 microliters (uL). In some embodiments, the detection array comprises at least about 6,144 positions. In some embodiments, positions of the detection array have a volume of less than about 50 microliters (uL). In some embodiments, the reaction products are dried on the detection array.

In another aspect, provided herein is a method for quantifying an amount of cellular proliferation. The method can include centrifuging a plate containing an array of cell cultures to provide an array of cell pellets in a solution; optionally removing the solution from the plate; imaging the plate to provide an image; from the image, detecting data associated with the cell pellets, which data comprises a location of each cell pellet; and quantifying a mass or number of cells in each cell pellet using the data associated with the cell pellets. The method can be capable of quantifying a mass or number of cells in at least 96 cell pellets from a single image.

In some embodiments, the solution is removed from the plate. In some embodiments, substantially all of the cells in the cell cultures are in the cell pellets. In some embodiments, the method further comprises reconstituting the cell pellets. In some embodiments, the method further comprises performing a subsequent procedure on substantially all of the cells.

In some embodiments, the method further comprises processing the image prior to detecting data associated with the cell pellets, wherein said processing comprises at least one of cropping the image, aligning the image, or thresholding the image.

In some embodiments, the solution is growth media. In some embodiments, the imaging is performed using a flat-bed document scanner. In some embodiments, the scanner is modified to keep a background constant and/or to put the plate in a location on scanner. In some embodiments, the imaging is performed using a camera. In some embodiments, the imaging uses the visible spectrum. In some embodiments, the cell pellets are not modified to over-express a fluorescent protein. In some embodiments, the data associated with the cell pellets includes a size of the pellet and/or a brightness of the pellet.

In some embodiments, a sample of about 900 pixels are used to the mass or number of cells. In some embodiments, a plurality of pixels of the image are scored on a scale of brightness ranging from 0 to 255. In some embodiments, said comparing is performed with the assistance of a trained machine learning algorithm. In some embodiments, the machine learning algorithm is trained on images acquired from cell pellets having a known mass or number of cells. In some embodiments, quantifying a mass or number of cells in each cell pellet does not include comparing an image of a cell pellet with a reference image that was generated at substantially the same time as the image of the cell pellet to be quantified.

In another aspect, provided herein is a method for high-throughput automated expression and functional screening of proteins. The method can comprise providing a colony plate having colonies of cells dispersed thereon, wherein the cells are capable of expressing one of a plurality of proteins; robotically inoculating a plurality of cultures from the colonies of cells, wherein the cultures are arrayed in wells of a culture plate; incubating the culture plate at conditions suitable for growing the cells in the cultures; robotically harvesting the cells from the cultures to provide cell pellets; imaging the cell pellets and using the image and a trained machine learning algorithm to quantify a mass or quantity of cells in the cell pellets; robotically combining each of the plurality of proteins from the cell pellets with a substrate in a reaction array; incubating the reaction array to produce a plurality of reaction products, which incubation is at conditions that are suitable for the proteins to transform the substrate into a reaction product; robotically transferring the reaction products to a detection array; and detecting the reaction products on the detection array. In some cases, a single person is capable of detecting at least 10,000 reaction products in a day.

In another aspect, provided herein is a system for high-throughput automated expression of a plurality of proteins. The system can comprise a reagent dispenser configured to dispense media into wells of a culture plate; a colony picker configured to pick colonies of cells from a colony plate and inoculate a plurality of cultures in wells of the culture plate; an incubator configured to incubate the culture plate at conditions suitable for growing the cells; a harvester configured to enrich the cells from the culture plate to provide cell pellets; and a plate transfer module configured to move the culture plate between the reagent dispenser, the colony picker, the incubator, and the centrifuge, where the system is capable of incubating at least 2,000 cultures in a day.

In some embodiments, the system further comprises a plate sealer configured to seal the wells of the culture plate from an atmosphere and a plate peeler configured to remove a seal applied by the plate sealer. In some embodiments, the harvester comprises a centrifuge. In some embodiments, the system further comprises a freezer.

In some embodiments, the reagent dispenser is further configured to dispense media into wells of a second culture plate; and inoculate the wells of the second culture plate with incubated first cultures to provide a plurality of second cultures; the plate sealer is further configured to seal the wells of the second culture plate from an atmosphere; the incubator is further configured to incubate the second culture plate at conditions suitable for growing the cells; the centrifuge is configured to harvest the cells from the second culture plate to provide cell pellets; the plate peeler further configured to remove a seal applied by the plate sealer; and the plate transfer module further configured to move the second culture plate between the reagent dispenser, the colony picker, the plate sealer, the incubator, the centrifuge and the plate peeler.

In some embodiments, a person does not physically contact the culture plate or the colony plate other than to provide empty consumable plates to the system and/or remove waste plates from the system.

In another aspect, provided herein is a system for high-throughput automated functional screening of proteins. The system can comprise an incubator configured to incubate a plurality of proteins with a substrate on a reaction array to produce a plurality of reaction products; a detection module configured to detect the reaction products on a detection array; one or more liquid handlers configured to lyse cells containing the protein, aliquot the protein onto the reaction array, aliquot the substrate onto the reaction array, and/or transfer some of the reaction products from the reaction array to the detection array; and a plate transfer module configured to move the reaction array and/or the detection array between the incubator, the detection module, and the one or more liquid handlers. The system is capable of detecting at least 10,000 reaction products in a day.

In some embodiments, a person does not physically contact the reaction array or the detection array other than to provide empty arrays to the system and/or remove waste arrays from the system.

In another aspect, provided herein is a system for high-throughput automated functional screening of proteins. The system can comprise a colony picker configured to pick colonies of cells from a colony plate and inoculate a plurality of cultures in wells of a culture plate; one or more incubators configured to incubate the culture plate at conditions suitable for growing the cells and expressing a protein in the cells, and incubate a plurality of proteins with a substrate on a reaction array to produce a plurality of reaction products; a harvestor configured to harvest the cells from the culture plate to provide cell pellets; a detection module configured to detect the reaction products on a detection array; one or more liquid handlers configured to dispense media into wells of a culture plate, lyse cells containing the protein, aliquot the protein onto the reaction array, aliquot the substrate onto the reaction array, and/or transfer some of the reaction products from the reaction array to the detection array; and a plate transfer module configured to move the culture plate, the reaction array and/or the detection array between the one or more liquid handlers, the colony picker, the one or more incubators, the centrifuge and the detection module.

In some embodiments, the method further comprises a plate sealer configured to seal the wells of the culture plate and/or the reaction array from an atmosphere. In some embodiments, the method further comprises a plate peeler configured to remove a seal applied by the plate sealer. In some embodiments, the method further comprises a camera or scanner capable of imaging the cell pellets. In some embodiments, the method further comprises a scheduler configured to coordinate the components of the system. In some embodiments, the method further comprises a laboratory information management system (LIMS) configured to record and/or provide data associated with the system.

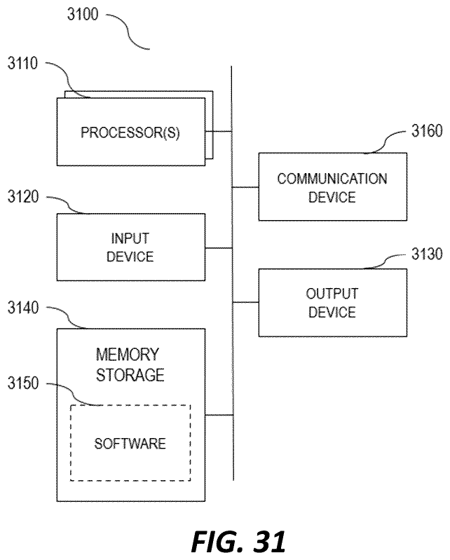

The operations described above are optionally implemented by components depicted in FIG. 31. It would be clear to a person having ordinary skill in the art how other processes are implemented based on the components depicted in FIG. 31.

FIG. 31 illustrates an example of a computing device in accordance with one embodiment. Device 3100 can be a host computer connected to a network. Device 3100 can be a client computer or a server. As shown in FIG. 31, device 3100 can be any suitable type of microprocessor-based device, such as a personal computer, workstation, server or handheld computing device (portable electronic device) such as a phone or tablet. The device can include, for example, one or more of processor 3110, input device 3120, output device 3130, storage 3140, and communication device 3160. Input device 3120 and output device 3130 can generally correspond to those described above, and can either be connectable or integrated with the computer.

Input device 3120 can be any suitable device that provides input, such as a touch screen, keyboard or keypad, mouse, or voice-recognition device. Output device 3130 can be any suitable device that provides output, such as a touch screen, haptics device, or speaker.

Storage 3140 can be any suitable device that provides storage, such as an electrical, magnetic or optical memory including a RAM, cache, hard drive, or removable storage disk. Communication device 3160 can include any suitable device capable of transmitting and receiving signals over a network, such as a network interface chip or device. The components of the computer can be connected in any suitable manner, such as via a physical bus or wirelessly.

Software 3150, which can be stored in storage 3140 and executed by processor 3110, can include, for example, the programming that embodies the functionality of the present disclosure (e.g., as embodied in the devices as described above).

Software 3150 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 3140, that can contain or store programming for use by or in connection with an instruction execution system, apparatus, or device.

Software 3150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 3100 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T31 or T3 lines, cable networks, DSL, or telephone lines.

Device 3100 can implement any operating system suitable for operating on the network. Software 3150 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

### EXAMPLES

### Example 1: Prediction of Enzyme Activity

The models described herein can predict enzymatic activity from sequence alone. A model not having a physics-bases simulation was trained on a dataset of about 17,000 enzyme variants based on a wild-type hydrolase backbone, screened against 9 substrates, built and tested by our robotic platform. The model was used to predict activity on variants that were measured but not included in the training dataset. The results achieved a sensitivity (true positive rate) of 0.89 and specificity (true negative rate) of 0.86. Such performance can form the foundation of a mapping of sequence space. Which can contribute to avoiding the experimental cost associated with non-active enzymes while not missing out on promising ones.

### Example 2: Extrapolation to Make Predictions

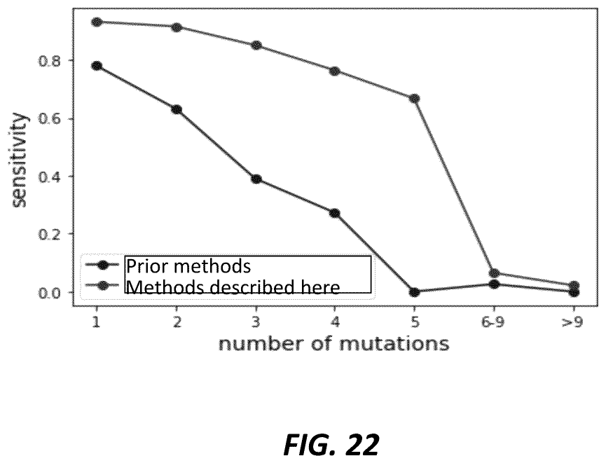

The models described herein can extrapolate to unseen parts of sequence space. When the same model was trained only on variants with 2 or fewer mutations (for about 100,000 variants) and made to predict on datasets of variants with different numbers of mutations that were measured but not in the training dataset, the model was still able to predict on variants with a higher number of mutations. FIG. 22 shows how the models described herein extrapolates to all variants with different numbers of mutations further away from the wild type, compared to an industry standard. This can make it possible to map and search over large swathes of sequence space to find the optimal solution, even if it does not exist within the bounds of the initial dataset. The further out the models can predict, the fewer experimental rounds are needed, and the faster and more cheaply the optimization objective can be reached.

### Example 3: Prescriptive Model

A prescriptive model was validated using in-silico simulations and lab measurements. With a gradient-based prescriptive model, in a single iteration, enzymes were designed and measured with more than 3-fold improvement over the wild-type (WT) enzyme for the target reaction. The active fraction of the library increased from 15% to 45%.

To increase the speed of our prescriptive models, a metamodel and cloud-distributed computing are used. This can improve an ability to prescribe new enzymes in days instead of months or years. Distributed computing clusters with hundreds of GPUs have been deployed and utilized with about 99% parallel efficiency.

### Example 4: Cell Growth

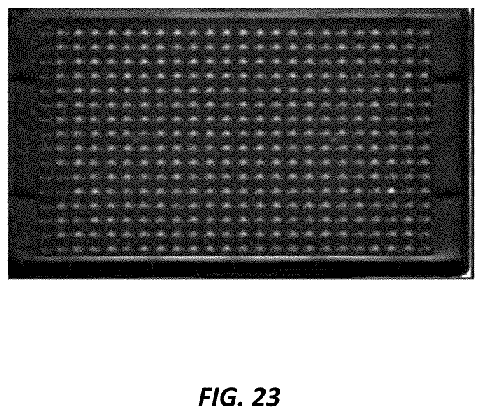

FIG. 23 shows a typical 384 well plate with cell pellets that contain enzymes expressed according to the methods described herein.

In some cases, cell growth in 384 well plates can pose a few challenges, mainly around culture oxygenation rates that could result in poor and non-uniform growth across the plate, thus leading to non-uniform biomass and enzyme expression. Additionally, the low culture volume in 384 well plates can make it difficult to detect enzyme activity.

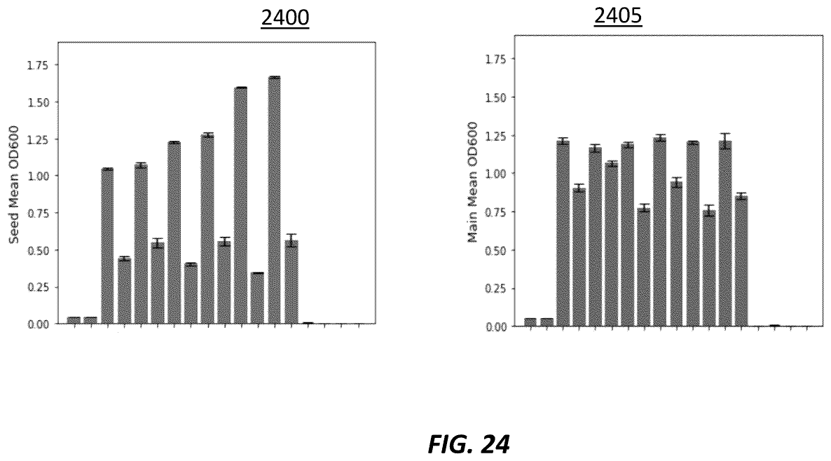

FIG. 24 demonstrates seed culture OD's 2400 and the resulting main OD's 2405 at various shake speeds and well volumes for seed growth. The error bars here represent standard errors. Lower volumes correspond with an increase in OD, likely due to improved oxygenation. In addition, shake speeds of 750 RPM increase final OD by about 20% when compared to seed cultures grown at 1,000 RPM. Without being held to any particular theory, the present disclosure surprisingly determined that a lower shaking speed allowed improved culturing, e.g., by better fitting an existing oxygen level to a reduced growth rate (i.e., better utilizing oxygen).

The growth is performed in incubators with 1 mm throw shakers. Bacteria can be grown to sufficient density to get enzymatic signals in 384 well plates. The enzyme levels produced are sufficient for activity assays in 1536 well plates and the culture density is sufficient for DNA sequencing by Sanger or next generation sequencing (NGS) methods

### Example 5: Cell Quantification

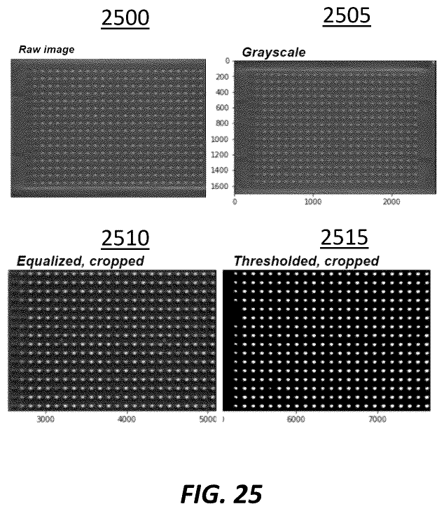

The cells are grown and pelleted according to the methods described herein. The spent media is discarded and an image of the pelleted cells in the plate is taken. FIG. 25 shows a series of processing of the raw image 2500. The image is converted to grayscale 2505, then is cropped and equalized 2510 (i.e., the image background is adjusted to the same shade). Detection of the pellet locations (as opposed to quantification of the quantity of cells) can benefit from a binary black/white 2515 rendition of the image.

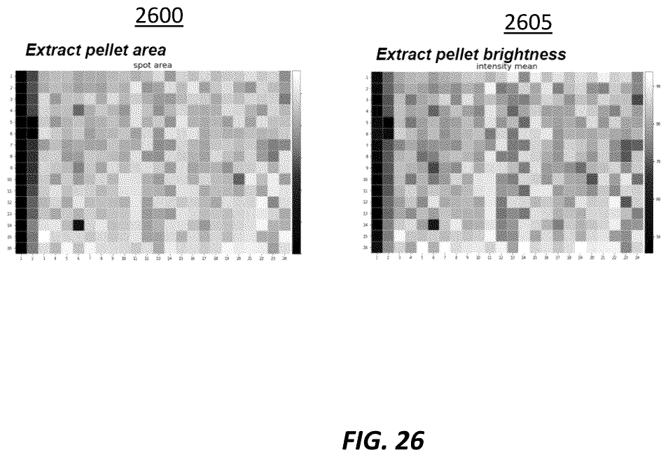

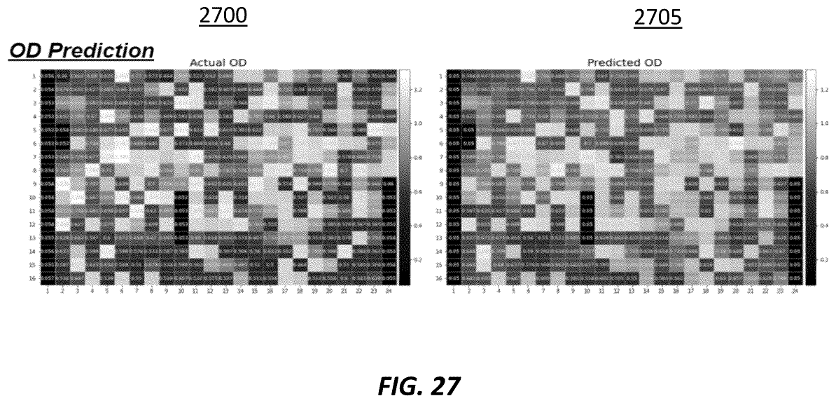

Continuing with FIG. 26, the area of the pellet 2600 can be extracted from the processed image. The brightness 2605 can be extracted and expressed in an intensity mean. This can involve pixelating the image as described herein. Continuing with FIG. 27, the actual OD 2700 correlates closely with the OD measured with the image analysis method described herein 2705.

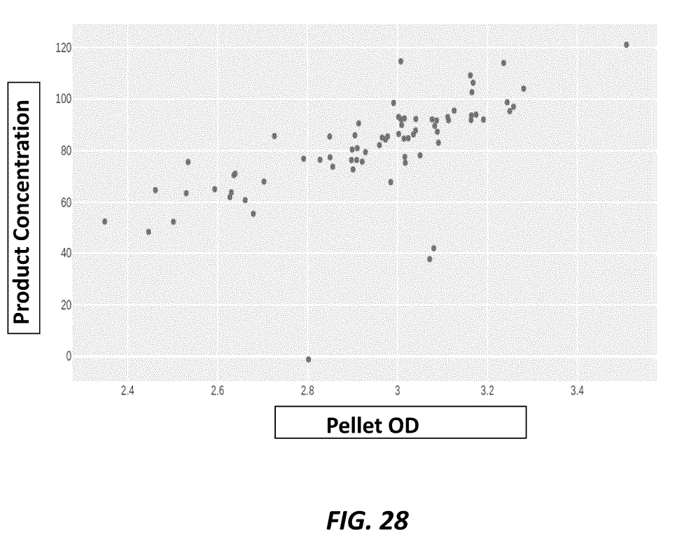

As seen in FIG. 28, there is a linear correlation between the amount of culture growth (Pellet OD) and the measured activity (vertical axis) for a given protein.

### Example 6: Enzyme Expression and Screening

The expression and screening system described herein was used. Starting with mutagenesis primers, gene fragments were constructed produce the targeted variants for the library. The mutagenesis library was cloned and transformed, and mutants were then seeded into trays and grown overnight to begin enzyme expression. This was followed by dilution and another growth cycle. The variant colonies were then cherry-picked to start a seed culture, and the cells then transferred to a main culture plate for a final overnight growth.

To prepare the enzymes needed for screening, the cell pellets grown in the main culture plates were washed and lysed using a lysis buffer. Enzyme samples were aliquoted into wells of a reaction plate, and the relevant substrate dispensed into each well. The reaction samples were then incubated to allow the enzymes to act on the substrates. Finally, the reacted samples were spotted onto target plates for LDI mass spec measurement. Detection plates had 1536 spots or 6144 spots. The measured spectra were processed with the analytics systems described herein.

These generate high throughput experimental data for reaction samples comprising enzyme and substrate molecules. A dataset of about 54,000 unique reaction samples where generated, including about 17,000 unique enzyme variants based on a single wild-type backbone of amidase reacted against nine substrate compounds, including L-prolinamide, ethyl 1-[2,5-dioxo-1-(4-propoxyphenyl)-3-pyrrolidinyl]-4-piperidinecarboxylate, and methyl 1-[2-(2,4-dichlorophenoxy)propanoyl]-4-piperidinecarboxylate.

A mass spectrum collected from one of the samples in the full dataset contains peaks from multiple different molecules present in the sample, including the substrate molecule, and the product molecule resulting from catalysis by the enzyme. Peak detection was performed on the spectra from all the samples in each plate. A heatmap shows the distribution of detected product peaks across all the samples in a 32×48 array plate.

### Example 7: Improvement of Enzyme Activity assisted by Computational Methods

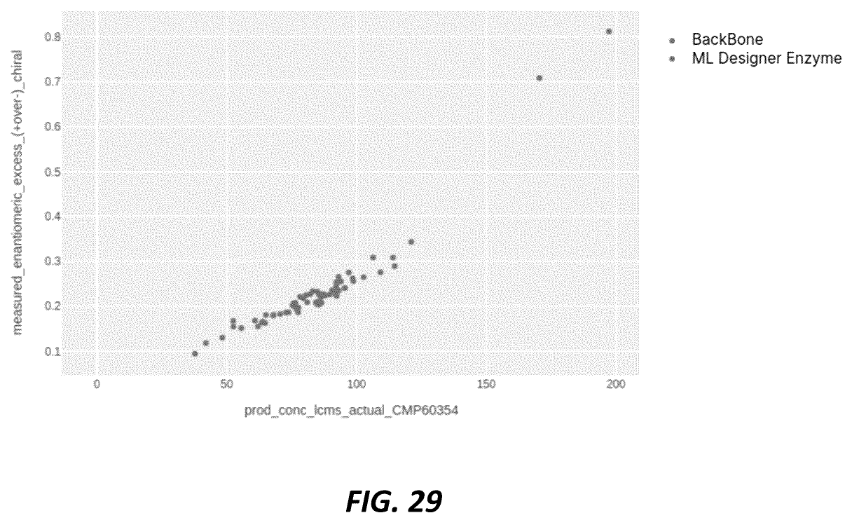

In FIG. 29, the vertical (y) axis is activity and the horizontal (x) axis is enantioselectivity. The blue points represent the previously best enzyme, which were used as a backbone for the search algorithm. The red points are the best enzymes designed by the algorithms described herein (i.e., having a combination of predictive models and search (prescriptive) algorithms). A single iteration the algorithms have improved both enantioselectivity and activity by a factor greater than two.

These results can demonstrate the results of a single machine learning-driven design cycle. The backbone was generated from a combination of machine learning and manual driven design iterations. Even starting from a fairly active enzyme the multi-objective optimization algorithm (coupled with the predictive model) was able to design an enzyme that was markedly improved in a single iteration.

### Example 8: Novel Activity Generation

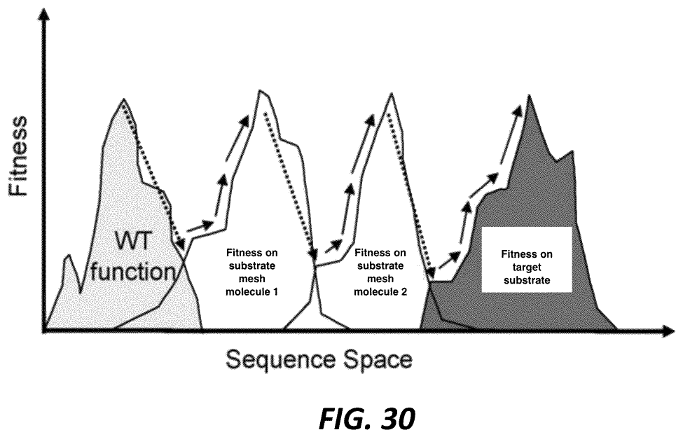

FIG. 30 shows a prophetic example of novel activity generation by a sequence-space walk. Here, a wild-type enzyme having no activity on a target substrate can be engineered to have activity by performing the methods described herein on a series of successive intermediate substrates (in this case two) having increased structural similarity to the target substrate.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having”, “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

1. A method for enzyme design, the method comprising:
proposing a plurality of candidate proteins using a prescriptive model that comprises a multi-objective search and optimization algorithm;
for each of the plurality of candidate proteins, predicting an enzymatic activity on a substrate using a predictive model that combines a machine learning algorithm and a physics-based simulation;
selecting and expressing at least some of the candidate proteins; and
measuring an activity on the substrate for the expressed candidate proteins.

2. The method of claim 1, further comprising using the measured activity to propose another plurality of candidate proteins using the prescriptive model.

3. The method of claim 1, further comprising using the measured activity to train or improve the prescriptive model.

4. The method of claim 1, further comprising using the measured activity to train or improve the machine learning algorithm of the predictive model.

5. The method of claim 1, wherein the candidate proteins are selected for expression based at least in part on the predicted activity on the substrate.

6. The method of claim 1, wherein the selected candidate proteins are expressed in E. coli.

7. The method of claim 6, wherein a quantity of E. coli that is cultured is measured using image analysis of pelleted E. coli cells.

8. The method of claim 6, wherein the E. coli are lysed to release the expressed proteins.

9. The method of claim 8, wherein the expressed proteins are enriched.

10. The method of claim 6, wherein the expressed proteins are contacted with the substrate.

11. The method of claim 10, wherein a concentration of the substrate and/or a reaction product are measured to determine an activity.

12. The method of claim 1, wherein an activity for each candidate protein is predicted and measured for a plurality of substrates.

13. The method of claim 12, wherein at least one of the plurality of substrates is a desired substrate.

14. The method of claim 12, wherein at least two of the plurality of substrates have substantially similar structures.

15. The method of claim 12, wherein at least two of the plurality of substrates have substantially dis-similar structures.

16. A method for evaluating a candidate protein, the method comprising, by one or more computing devices:
obtaining an amino acid sequence associated with the candidate protein;
inputting the amino acid sequence into a predictive model to obtain a predicted activity on a substrate, wherein the predictive model comprises a trained machine learning model and a physics-based simulation model; and
evaluating the candidate protein based on the predicted activity.

17. (canceled)

18. The method of claim 16, wherein the amino acid sequences associated a plurality of candidate proteins are obtained using a prescriptive model and input into the predictive model.

19. The method of claim 18, wherein the prescriptive model comprises a multi-objective search and optimization algorithm.

20. The method of claim 18, wherein said evaluating comprises selecting one or more candidate proteins based at least partially on the predicted activity.

21. The method of claim 20, wherein the one or more candidate proteins are selected for measurement of activity.

22.-24. (canceled)

25. The method of claim 16, wherein machine learning model is configured to be trained using a combination of stochastic and deterministic optimization methods.

26.-37. (canceled)

38. The method of claim 16, wherein the physics-based model is configured to compute enzyme and substrate relative positions.

39. (canceled)

40. The method of claim 16, wherein the physics-based model constrains the machine learning model of the predictive model to a physical solution space.

41.-45. (canceled)

46. The method of claim 16, wherein machine learning model is configured to predict a folded structure for the candidate protein based on the amino acid sequence of the candidate protein.

47. (canceled)

48. A method for identifying one or more candidate proteins, the method comprising, by one or more computing devices:
obtaining a plurality of candidate proteins using a prescriptive model comprising a multi-objective search and optimization algorithm;
inputting each candidate protein of the plurality of candidate proteins into a predictive model to obtain a predicted activity on a substrate; and
selecting one or more candidate proteins from the plurality of candidate proteins at least partially based on the predicted activities.

49.-51. (canceled)

52. The method of claim 48, wherein the predictive model comprises a trained machine learning model and a physics-based simulation model.

53. The method of claim 48, wherein the one or more candidate proteins are selected for measurement of activity.

54.-63. (canceled)

64. The method of claim 48, wherein the prescriptive model is based at least partially on a combination of stochastic and deterministic optimization methods.

65.66. (canceled)

67. The method of claim 48, wherein the prescriptive model comprises a meta-model-assisted evolutionary algorithm.

68.-76. (canceled)

77. A method for obtaining one or more candidate proteins, the method comprising, by one or more computing devices:
receiving an initial set of candidate proteins; and
obtaining the one or more candidate proteins by performing, based on the initial set of candidate proteins, one or more iterations of an evolutionary algorithm which utilizes problem-specific evolution operators, each iteration comprising:
evaluating a current set of candidate proteins; and
based on the evaluation, updating the current set of candidate proteins.

78.-126. (canceled)
